SEQUENCE LISTING (61 sequences)

SEQ ID NO: 1 - protein, 627 aa, kluyveromyces marxianus
MSQLTAFLIDVAAGNSPLATPETTTQCLSYLEYTLLNKAHAQRKTDYVQVALANLSSNDPDRDPALPVNMAMLAGPQPRPLISVKDVGEWVGRVSRHLERIEDPKDEQDEETDVSSSLFNALLVLVLQLKEFVGKRKMRVRIVVFTGDTFQDVSSDELETFDQQCPFEVVLVSPSALPSALPKAETNDSGAKALSKAASLSHGLLFSTQQMISAIADPRPKLVRPVRIFEGQLRLGDPQLPESLCINVEAYPGTKQVSLESRKVLHRVGEQGLYEYKPVKSVVEYYTGPDGPNSDDTGATSGPSVSISSQYISKAYRYGSDYVSLPHLLEEKRQFHASPGIDIRGFLDMDKLERRYLCSESVYVVAGSTSRADYVGFCSLVDALAKQRRTVLARWVPKSGSEAQMCVLAPARGSNGERVLVMSRLAMAEEERGFSGAAASSEAAASPEAAGSDLLMERFVEGMTMQGGSSPGPGPGPIQRYADMAVDTGVPLPMDQTSGKSGQKSAHSLDLPPAIPLHLQRWAILHKVHTNYISQYLGLGLGQGPGNEQDPEGNSDSTLLSHRTPPMSPSVTRTFTPHHGPLELSHQLKSHLGVEKQPERRSTDSPAAPSTPQDPELLDLEALLGGA

SEQ ID NO: 2 - DNA, 1884 bp, kluyveromyces marxianus
atgtcccagctcacagcgtttctaattgacgttgcagcgggcaacagcccgctggcaacccccgagacaaccacccaatgcctctcgtacctcgaatatacccttctcaacaaggcccatgcccaaagaaagacagactacgtccaggtagcactcgcaaacctcagctcaaacgacccagaccgcgaccccgcgctgcccgtgaacatggcgatgctcgcgggcccacaaccaaggccattgataagcgtcaaggatgttggagagtgggtggggagggtgtccaggcatttggagaggattgaggaccccaaagatgagcaagatgaggagacggatgtctcctcatcgctgttcaatgcattgttagtgttggtactccaactcaaggagtttgttggtaagcgcaagatgcgcgtgcgcatcgtggtattcactggagacacgttccaagatgtgtctagcgatgagctggaaacgtttgaccagcagtgtccgtttgaggtggtactggtttctccctctgccctgccctctgccttgccaaaggcagagacgaatgattcaggggccaaggcccttagcaaggcagcatcgttgtcgcacggcctcctcttctccacacagcagatgatctctgccatcgcagatcccagaccgaaactcgtgcggcccgtgcgcatcttcgagggccagctacggctgggcgaccctcagctgcccgaatcgctctgtatcaacgtcgaggcgtaccctgggaccaagcaggtgtcgctagagtcgcgaaaggtgctgcacagggtgggggaacaaggcttgtacgagtacaagccggtgaaatcggtggtggagtactatacggggccagatgggcccaatagtgatgacactggggccactagtggccccagtgtgagtatctctagccagtacatctccaaagcctatcggtacggcagcgattacgtgtcgctccctcacctgctcgaagagaaacgccagttccatgcttccccaggtatcgatatcagagggtttctcgacatggataagctcgaaaggcggtacttgtgttcggagagcgtgtacgtggtggcgggaagtaccagcagggccgattacgttgggttctgctcgcttgtggatgcattggcgaagcagcgacgcacggttctggcgcgatgggtgcccaagtcgggcagcgaggcgcagatgtgcgtgctggcgccggctaggggcagtaatggggagcgtgtgcttgtgatgagccggctggcgatggctgaggaggagaggggctttagtggggcagctgcctcgtcagaggctgctgcctcgccagaggccgcaggctcggacttgctcatggagcggtttgtggagggcatgacgatgcaagggggctctagcccaggcccagggcctgggccaatccagaggtatgccgatatggctgtagacaccggagttccgctcccaatggaccaaacgtcgggcaagagtggacaaaagtctgcccatagtcttgacctgcccccagccatcccactgcatctccagcgctgggccatcttacacaaggtacacaccaactacatatcccaatacctgggccttggcctgggccaaggcccaggcaatgagcaggaccccgagggaaattctgacagcacccttttgtcccataggacccctccgatgagcccatccgtcacacgcacgttcacgccgcaccacggcccgctcgagctctcacaccagctcaagtcgcacctgggcgtcgagaagcagcccgaacggcgctctaccgactctccagctgctccatccactccccaggacccagaactgctcgatctagaggccttgttgggcggggcctag

SEQ ID NO: 3 - protein, 563 aa, kluyveromyces marxianus
MSEITLGKYLFERLKQVNVNTVFGLPGDFNLSLLDKIYEVEGMRWAGNANELNAAYAADGYARIKGMSCIITTFGVGELSALNGIAGSYAEHVGVLHVVGVPSISSQAKQLLLHHTLGNGDFTVFHRMSANISETTAMITDIATAPAEIDRCIRTTYVTQRPVYLGLPANLVDLNVPAKLLQTPIDMSLKPNDAESEKEVIDTILALVKDAKNPVILADACCSRHDVKAETKKLIDLTQFPAFVTPMGKGSIDEQHPRYGGVYVGTLSKPEVKEAVESADLILSVGALLSDFNTGSFSYSYKTKNIVEFHSDHMKIRNATFPGVQMKFVLQKLLTNIADAAKGYKPVAVPARTPANAAVPASTPLKQEWMWNQLGNFLQEGDVVIAETGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEVMLPVFDAPQNLVEQAKLTAATNAKQ

SEQ ID NO: 4 - DNA, 1692 bp, kluyveromyces marxianus
atgtctgaaattactttgggtaaatatttgttcgaaagattaaagcaagtcaacgttaacaccgttttcggtttgccaggtgacttcaacttgtccttgttggacaagatctacgaagttgaaggtatgagatgggctggtaacgccaacgaattgaacgctgcttacgccgctgatggttacgctcgtatcaagggtatgtcttgtatcatcaccaccttcggtgtcggtgaattgtctgctttgaacggtattgccggttcttacgctgaacacgtcggtgttttgcacgttgttggtgtcccatccatctcttctcaagctaagcaattgttgttgcaccacaccttgggtaacggtgacttcactgttttccacagaatgtctgccaacatttctgaaaccactgctatgatcactgacattgctaccgccccagctgaaattgacagatgtatcagaaccacttacgtcacccaaagaccagtctacttaggtttgccagctaacttggtcgacttgaacgtcccagctaagttgttgcaaactccaattgacatgtctttgaagccaaacgatgctgaatccgaaaaggaagtcattgacaccatcttggctttggtcaaggatgctaagaacccagttatcttggctgatgcttgttgttccagacacgacgtcaaggctgaaactaagaagttgattgacttgactcaattcccagctttcgtcaccccaatgggtaagggttccattgacgaacaacacccaagatacggtggtgtttacgtcggtaccttgtccaagccagaagttaaggaagccgttgaatctgctgacttgattttgtctgtcggtgctttgttgtctgatttcaacaccggttctttctcttactcttacaagaccaagaacattgtcgaattccactccgaccacatgaagatcagaaacgccactttcccaggtgtccaaatgaaattcgttttgcaaaagttgttgaccaatattgctgacgccgctaagggttacaagccagttgctgtcccagctagaactccagctaacgctgctgtcccagcttctaccccattgaagcaagaatggatgtggaaccaattgggtaacttcttgcaagaaggtgatgttgtcattgctgaaaccggtacctccgctttcggtatcaaccaaaccactttcccaaacaacacctacggtatctctcaagtcttatggggttccattggtttcaccactggtgctaccttgggtgctgctttcgctgctgaagaaattgatccaaagaagagagttatcttattcattggtgacggttctttgcaattgactgttcaagaaatctccaccatgatcagatggggcttgaagccatacttgttcgtcttgaacaacgatggttacaccattgaaaagttgattcacggtccaaaggctcaatacaacgaaattcaaggttgggaccacctatccttgttgccaactttcggtgctaaggactacgaaacccacagagtcgctaccaccggtgaatgggacaagttgacccaagacaagtctttcaacgacaactctaagatcagaatgattgaggttatgttgccagtcttcgatgctccacaaaacttggttgaacaagctaagttgactgctgctaccaacgctaagcaataa

SEQ ID NO: 5 - protein, 578 aa, kluyveromyces marxianus
MEYADRYNLEPLIPLAEYLFHRLFQLNCHTVFGVPNYSTAKLYGALAATGIQWIQTINQLNTSFAADAYGRTIGISCYITSESAELAHINGFFGSYCEYVPILQLVILEHSHDLERLIGDVSIFHDIVDDPAEIDFGLRTLFWGKRPVYMGLRSKDISRLVPSIALNQSILNKETPAPFPSSLSLSIARYQQRQQEQSIVGDIVDQILSKLYSCSTPIIVVDALIDRYNYNDMLQNFLAETGIPFVTTLMSKAAINESLPNFIGTFLGTLSHPTVREYMNNSDCTLILGCVIDNFKNSYCRFSYKNKCQIMLWNDRVKIENNLIPDVPIHEILPQLIAKIDASKLSNLYAVTVPDMIPRVEPKPVTFLRQEYLWFRMSTWLKEGDVIISESGTSAIGLLRQKFPDNSRLVSQTIWNSSGYSIGACLGVLTAYRDLGKLDKHRIILIVGDGSLQFTFQELSTILTQGFEPYIYVVNNQGYTVDRTLNKEKTHTNATYFDIQQWEILSIPSLFNSRDYFKRKCMTVGELNSLLNDEEFNDPRRLKIVELILPSMDVPVLLEPHDDSSDDELTPQSKRVRL

SEQ ID NO: 6 - DNA, 1737 bp, kluyveromyces marxianus
atggagtatgctgataggtacaacttggagcctcttataccgctcgcagagtaccttttccacaggttattccaactgaattgtcatacggtatttggagttccaaattattcaacagcaaaactgtatggtgcccttgcagccactggaattcaatggattcagactattaaccaattgaatacgtcatttgctgctgatgcatatggtaggactattggcataagttgctacataactagcgaatctgcggaattggcacatattaacggattttttggctcctattgcgagtacgtgccgattttacagttggttatattagagcactctcatgacctagaaaggctcattggtgatgtatccatatttcatgatattgtggatgatcctgcagaaatagatttcggacttcgaacgctgttttggggtaagagacccgtttacatgggtcttcgatctaaagatatttcaagattagtgccaagcattgctctaaaccagagtatattaaataaagaaacaccggcgccctttccctcatctctttctctctcgattgctcggtaccagcagaggcagcaagaacagagcattgttggggacatagttgatcagatattgtccaagctatactcttgttctactccaataatagtggttgatgcactgattgatagatacaattacaatgacatgttgcaaaacttccttgcggaaacaggaataccttttgtaactacgctaatgtccaaggctgctatcaatgaaagtttgcctaactttatcggcactttcttggggacgttatcgcaccctacagttcgtgaatacatgaataattcagattgtacgcttatactgggttgtgtgattgacaattttaagaattcgtactgtaggttttcgtacaaaaacaagtgccagatcatgctatggaatgacagagtcaagatagaaaacaatttaattccagatgttcctattcatgagattcttcctcagcttatagcaaaaattgacgcatcaaagctctctaatctgtacgccgtcacagttcccgatatgattccaagagttgaacccaagccggttacttttctcagacaggagtacctatggttcagaatgtccacttggttaaaagaaggcgatgttatcatttctgaatccggtacatccgcgatcggcttgttgcgtcagaaattcccggataattcaaggcttgtatcccagaccatctggaattcttcaggatactccattggagcttgccttggtgtcctgacagcgtatagagatttggggaagctggacaagcatcggatcattttgatcgtaggtgatggttcgctacaattcactttccaagaattgagtaccattctcactcaaggttttgaaccgtacatctacgtagtgaataaccaaggttataccgtcgacagaaccttgaacaaggaaaagactcacacgaatgccacatacttcgatattcaacaatgggaaatactcagcataccgtcacttttcaattcaagagattacttcaagagaaaatgcatgaccgttggagaattgaacagcctcttgaatgatgaagaattcaatgacccgcgacggctcaagatagtcgaattgattcttccttctatggatgttcctgttctccttgaacctcatgacgatagtagtgatgatgaacttactcctcaaagcaaaagagtaagactttag

SEQ ID NO: 7 - protein, 362 aa, kluyveromyces marxianus
MSKNIVVLPGDHVGTEITNEAIKVLNAISEARPSIKFNFEHHLIGGAAIDATGVPLPDEALEASKKADAVLLGAVGGPKWGTGSVRPEQGLLKIRKELGLYANLRPCNFASDSLLDLSPLKPEYAKGTDFVVVRELVGGIYFGERKEDEGDGVAWDSEKYSVPEVQRITRMAAFLALQHNPPLPIWSLDKANVLASSRLWRKTVEETIKNEFPQLTVQHQLIDSAAMILVKSPTKLNGIVITNNMFGDIISDEASVIPGSLGLLPSASLASLPDTNKAFGLYEPCHGSAPDLPANKVNPIATILSAAMMLKLSLDLVEEGRAVEEAVRKVLDSGIRTGDLGGSNSTTEVGDAVAKAVKEILA

SEQ ID NO: 8 - DNA, 1089 bp, kluyveromyces marxianus
atgtcaaagaatatcgttgtcttaccaggtgatcatgtcggtactgaaattaccaatgaagcgatcaaagttttgaacgctatttcagaagcacgcccttctatcaagtttaattttgaacatcatttgattggtggtgctgcaattgatgctactggtgtgccattgccagacgaagctttggaagcttccaagaaagctgatgcagtattattgggtgcagttggtggtccaaagtggggtacaggttccgttagacctgaacaaggtttattgaagatcagaaaggaattgggattatacgctaacttgaggccttgtaattttgcttcagactctttgcttgatctatctccattgaaacctgagtacgccaagggaactgactttgttgtcgttagagaattggtgggtggtatctactttggtgagaggaaggaagatgaaggtgatggtgttgcttgggattctgaaaagtacagtgtaccagaagttcaaagaatcaccagaatggctgcatttttagctttacaacacaacccacctttaccaatctggtcattggataaagcaaatgtcttggcatcatctagattgtggagaaaaacagtagaagaaaccatcaagaatgagttccctcaattgactgtccaacatcaattgatcgattccgctgctatgatcctcgtcaagtctccaaccaagttaaatggtatagttattacgaataacatgtttggtgatatcatttctgatgaagcttccgtcattccaggttcattgggcttactaccatccgcttctttggcctcgctaccagacaccaacaaagcattcggtttgtacgaaccatgtcacggttctgcacccgacttaccagcaaataaggtcaacccaattgcaacaattctatctgcagctatgatgttaaaactttccctagatctagtggaagaaggtagagccgttgaagaagccgttagaaaagttttggattctggaatcagaactggcgatctaggcggttccaactctaccacggaggttggtgacgctgttgcaaaggctgtaaaggagattttggcatag

SEQ ID NO: 9 - DNA, 27 bp, Artificial Sequence
  Synthetic forward primer KmKU80_1F_Xho
gcactcgagtttgcaccaggccttgtc

SEQ ID NO: 10 - DNA, 27 bp, Artificial Sequence
  Synthetic reverse primer KmKU80_2B_Eco
actgaattcaaacgctgtgagctggga

SEQ ID NO: 11 - DNA, 1055 bp, kluyveromyces marxianus
gcactcgagtttgcaccaggccttgtcccaggccttggcctggggcagggcaattgctcatgctctcgccctggctgactcccaggccctggcctgggatgtcaaagtattcgggaactgctttgaacgtgggcatggtggataccttggtgtatgcctatgcttggcctatgccttggccaaggtcttgttagagtctgtttaacagtgggaaagtcaattagaatgaatattcgattatataggatgagtagatgaatagatcaataaataaggaggataatgattaggcaaagcctaggcctatcggcctggcttcgcctggcctggcttcgccttcagaagttcaccttgttgttgggctcgcctctgggcgtctctggatgcatcttgtagtacatcgcctcgaactcttctcgcgagagtttggaaacgtccaccatctgaggttgggcctgggcctgaccgtcctcctggctcctgggcaccgcttggaccttgttggggcctctatactctctggcaaacatataggtggtgtttcccgtgccaatattggagtggtttccgcctctggacttggcgtcgcctggggcgtgcttgtttcggttgtagacttcgaacgcttcgaagaacttggacattgtattagtagttgcgtgcagcttgctgcatgcgactgccaataggagagagaaggtctaaccatcccaagctgaggccttggccccagcttgtctttatataaggtactagataatacatagggcttgtcaatgactaaggcaagggataaacaagggatactcaagggatactaaagggatgccccttgacaaggggcaacaataccccagggatacccctttggtacttaattgctgacttgttgctgacttatcagtaaccaccttaattaaggccaggcctactgattatagcatcagcttctccaagaaaagagagaccccaagaacaaggagaaagacctcccaggagacaggagaaggacctcccgcaaggccatgtcccagctcacagcgtttgaattcagt

SEQ ID NO: 12 - DNA, 28 bp, Artificial Sequence
  Synthetic forward primer KmKU80_3F_Bam
agtggatccctaattgtatacctggggc

SEQ ID NO: 13 - DNA, 28 bp, Artificial Sequence
  Synthetic reverse primer KmKU80_4B_Sac
tgagagctcgtcgaggaattgcttgatc

SEQ ID NO: 14 - DNA, 1052 bp, kluyveromyces marxianus
agtggatccctaattgtatacctggggcaatgtataactggggccaaggccaaggccaaggccatgtataactgtataatagaatagacaagcagggatacggcaatccattggcatggcaatggccatggcattgggatggcatcggtcctgccccatgcttcgctgcgcttcgcatggggcatcgcatgggcatcgcatggctcctcggaccactgtcttccccggccttgtctcgtggtccgtcgaaaacccaactaatataaaagggaaactatcaccacaagaagaacacagaagtaacacccacaattaatacacactaccccaggataaccccaggtactagccagacacaagccagatactagcagtgtcaacaatatcccccaagcattcagataacaacatacagcatgactatcgtcaacatccccaccaaaccctaccaggaccagaaaccaggcacttcgggcttgagaaagaagaccaaggtcttcatggaacagccccactacaccgaaaacttcatccaggccatcatggaagccatcccagaaggttcccaaggcgccactttggtcattggtggcgatggccgctactataacgacaaagtcgtccagttgatcgctgccattggctccgccaatggcgttcgcaaactcattatcggccacaacggtatcctctccacgccagctgcctccaacgtcatcagaacgtaccacgaaaagtgtaccggtggtatcatcttgaccgcctcgcacaatccaggtggtccaaccaacgatttcggtatcaagtacaacttggccaacggtgggccagccccagaatcggtcacaaacaagatctgggagcaatccaagcaattgacctcctacaagatcgtcgagaacttcccgcaattggacttgtccaagttgggcgagaaccagaagtacggcgacttgctcgtggacgtgatcgactccactgctgcctacacgcaattgatgaaacgcatcttcgatttcccattgatcaagcaattcctcgacgagctctca

SEQ ID NO: 15 - DNA, 5956 bp, kluyveromyces marxianus
tcgagtttgcaccaggccttgtcccaggccttggcctggggcagggcaattgctcatgctctcgccctggctgactcccaggccctggcctgggatgtcaaagtattcgggaactgctttgaacgtgggcatggtggataccttggtgtatgcctatgcttggcctatgccttggccaaggtcttgttagagtctgtttaacagtgggaaagtcaattagaatgaatattcgattatataggatgagtagatgaatagatcaataaataaggaggataatgattaggcaaagcctaggcctatcggcctggcttcgcctggcctggcttcgccttcagaagttcaccttgttgttgggctcgcctctgggcgtctctggatgcatcttgtagtacatcgcctcgaactcttctcgcgagagtttggaaacgtccaccatctgaggttgggcctgggcctgaccgtcctcctggctcctgggcaccgcttggaccttgttggggcctctatactctctggcaaacatataggtggtgtttcccgtgccaatattggagtggtttccgcctctggacttggcgtcgcctggggcgtgcttgtttcggttgtagacttcgaacgcttcgaagaacttggacattgtattagtagttgcgtgcagcttgctgcatgcgactgccaataggagagagaaggtctaaccatcccaagctgaggccttggccccagcttgtctttatataaggtactagataatacatagggcttgtcaatgactaaggcaagggataaacaagggatactcaagggatactaaagggatgccccttgacaaggggcaacaataccccagggatacccctttggtacttaattgctgacttgttgctgacttatcagtaaccaccttaattaaggccaggcctactgattatagcatcagcttctccaagaaaagagagaccccaagaacaaggagaaagacctcccaggagacaggagaaggacctcccgcaaggccatgtcccagctcacagcgtttgaattcagatcttccagtggtgcatgaacgcatgagaaagcccccggaagatcatcttccgggggctttttttttggcgcgcgatacagaccggttcagacaggataaagaggaacgcagaatgttagacaacacccgcttacgcatagctattcagaaatcaggccgtttaagcgatgattcacgagaattgctggcccgctgcggcataaaaattaatttacacactcagcgcctgattgcgatggcggaaaacatgccgattgatatcctgcgcgtgcgtgatgatgacattccgggtctggtaatggatggcgtggtcgatctcggtattatcggcgaaaacgtgctggaagaagagctactcaaccgccgcgcacagggcgaagatccacgctatttaaccctgcgccgtcttgacttcggcggctgccgtttatcgctggcaacaccggttgacgaagcctgggacggcccggccgcgctggacggtaaacgtatcgctacctcatatccgcacctcctcaaacgctacctcgaccagaaaggcgtctcttttaaatcgtgtctgttaaatggttctgtcgaagtcgcgccgcgcgcggggctggccgacgctatctgcgatttggtctctaccggcgcgacgcttgaagctaacggcctgcgtgaagtcgaagttatctaccgctctaaagcctgtctgattcagcgcgacggtgagatggcacagagcaagcaagagctgatcgataaattgctgacccgtattcagggcgtgattcaggcgcgcgaatcgaaatacatcatgatgcacgcgccaagtgaacgcctggaagaggttatcgccctgctgccaggcgccgaaaggccgacaattctgccgctggcaggcgagcaacagcgcgtggcgatgcacatggtcagcagcgaaacgttgttctgggaaaccatggagaaactgaaagcgcttggcgccagctcgattctggtactgccgatcgagaagatgatggagtgatctgacgcctgatggcgctgcgcttatcaggcctacgtaatgcgttgatattttgggttctgtaggccggataaggcggaaccctgtgatggagtaaagaccatgagcttcaataccctgattgactggaacagggatctgaattaattctcatgtttgacagcttatcatcgataagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcttccagtggtgcatgaacgcatgagaaagcccccggaagatcatcttccgggggctttttttttggcgcgcgatacagaccggttcagacaggataaagaggaacgcagaatgttagacaacacccgcttacgcatagctattcagaaatcaggccgtttaagcgatgattcacgagaattgctggcccgctgcggcataaaaattaatttacacactcagcgcctgattgcgatggcggaaaacatgccgattgatatcctgcgcgtgcgtgatgatgacattccgggtctggtaatggatggcgtggtcgatctcggtattatcggcgaaaacgtgctggaagaagagctactcaaccgccgcgcacagggcgaagatccacgctatttaaccctgcgccgtcttgacttcggcggctgccgtttatcgctggcaacaccggttgacgaagcctgggacggcccggccgcgctggacggtaaacgtatcgctacctcatatccgcacctcctcaaacgctacctcgaccagaaaggcgtctcttttaaatcgtgtctgttaaatggttctgtcgaagtcgcgccgcgcgcggggctggccgacgctatctgcgatttggtctctaccggcgcgacgcttgaagctaacggcctgcgtgaagtcgaagttatctaccgctctaaagcctgtctgattcagcgcgacggtgagatggcacagagcaagcaagagctgatcgataaattgctgacccgtattcagggcgtgattcaggcgcgcgaatcgaaatacatcatgatgcacgcgccaagtgaacgcctggaagaggttatcgccctgctgccaggcgccgaaaggccgacaattctgccgctggcaggcgagcaacagcgcgtggcgatgcacatggtcagcagcgaaacgttgttctgggaaaccatggagaaactgaaagcgcttggcgccagctcgattctggtactgccgatcgagaagatgatggagtgatctgacgcctgatggcgctgcgcttatcaggcctacgtaatgcgttgatattttgggttctgtaggccggataaggcggaaccctgtgatggagtaaagaccatgagcttcaataccctgattgactggaacagccggatctggccggatccctaattgtatacctggggcaatgtataactggggccaaggccaaggccaaggccatgtataactgtataatagaatagacaagcagggatacggcaatccattggcatggcaatggccatggcattgggatggcatcggtcctgccccatgcttcgctgcgcttcgcatggggcatcgcatgggcatcgcatggctcctcggaccactgtcttccccggccttgtctcgtggtccgtcgaaaacccaactaatataaaagggaaactatcaccacaagaagaacacagaagtaacacccacaattaatacacactaccccaggataaccccaggtactagccagacacaagccagatactagcagtgtcaacaatatcccccaagcattcagataacaacatacagcatgactatcgtcaacatccccaccaaaccctaccaggaccagaaaccaggcacttcgggcttgagaaagaagaccaaggtcttcatggaacagccccactacaccgaaaacttcatccaggccatcatggaagccatcccagaaggttcccaaggcgccactttggtcattggtggcgatggccgctactataacgacaaagtcgtccagttgatcgctgccattggctccgccaatggcgttcgcaaactcattatcggccacaacggtatcctctccacgccagctgcctccaacgtcatcagaacgtaccacgaaaagtgtaccggtggtatcatcttgaccgcctcgcacaatccaggtggtccaaccaacgatttcggtatcaagtacaacttggccaacggtgggccagccccagaatcggtcacaaacaagatctgggagcaatccaagcaattgacctcctacaagatcgtcgagaacttcccgcaattggacttgtccaagttgggcgagaaccagaagtacggcgacttgctcgtggacgtgatcgactccactgctgcctacacgcaattgatgaaacgcatcttcgatttcccattgatcaagcaattcctcgacgagct

SEQ ID NO: 16 - DNA, 18 bp, Artificial Sequence
  Synthetic forward primer Iden_KU80BF
tgtgtctagcgatgagct

SEQ ID NO: 17 - DNA, 18 bp, Artificial Sequence
  Synthetic reverse primer Iden_KU80EF
aaaccgctccatgagcaa

SEQ ID NO: 18 - DNA, 18 bp, Artificial Sequence
  Synthetic forward primer Iden_KU80DS_1F
acaaggagaaagacctcc

SEQ ID NO: 19 - DNA, 19 bp, Artificial Sequence
  Synthetic reverse primer ScURA3_N_2B
gcagcacgttccttatatg

SEQ ID NO: 20 - DNA, 18 bp, Artificial Sequence
  Synthetic forward primer ScURA3_C_1F
agaagatgcggccagcaa

SEQ ID NO: 21 - DNA, 18 bp, Artificial Sequence
  Synthetic reverse primer Iden_KU80DS_2B
gccccagttatacattgc

SEQ ID NO: 22 - DNA, 21 bp, Artificial Sequence
  Synthetic forward primer ScURA3_1F_47
atgtcgaaagctacatataag

SEQ ID NO: 23 - DNA, 18 bp, Artificial Sequence
  Synthetic reverse primer ScURA3_2B_43
tgtgcattcgtaatgtct

SEQ ID NO: 24 - DNA, 18 bp, Artificial Sequence
  Synthetic forward primer Iden_His-G_1F_48
atacagaccggttcagac

SEQ ID NO: 25 - DNA, 18 bp, Artificial Sequence
  Synthetic reverse primer Iden_His-G_2B_48
ttatccggcctacagaac

SEQ ID NO: 26 - DNA, 18 bp, Artificial Sequence
  Synthetic forward primer Iden_KU80DS_1F
acaaggagaaagacctcc

SEQ ID NO: 27 - DNA, 18 bp, Artificial Sequence
  Synthetic reverse primer Iden_KU80DS_2B
gccccagttatacattgc

SEQ ID NO: 28 - DNA, 27 bp, Artificial Sequence
  Synthetic forward primer KmPDC1_5D_1F_Xho
gatctcgagccgaaagatcgtccgatt

SEQ ID NO: 29 - DNA, 30 bp, Artificial Sequence
  Synthetic reverse primer KmPDC1_5D_2B_Bgl
ctaagatcttgcaattatttggtttgggtg

SEQ ID NO: 30 - DNA, 1036 bp, kluyveromyces marxianus
gatctcgagccgaaagatcgtccgattatccagcgaatatacagcgtgaataatggaatggccttgtattcgttttttccgagagaaaaaaacgggcttcggtgaaaatcgggtgaatatgcaactagcgggacgaatgctctggaaatgcatatcctatgcaactagcgggatgaacaaatctcaccccagaattcgcaggaaaaaacaggaaaaaaaaaaagaaggccaccacggccacaaagaccacaaagaccacaaaaaaaaacaaaaaacaaccgtcccagcttccagtgtttggaatactggaacacaggaagccgcataagagtgggcgttgcacaggaagccaggcccagaagccccagagttacttttttttttttgttttttccttctgttcgctgtgcccgcatcagatgatgcgcctttatttacgatgccaatgcgaatagcaccagtgagagcaccagtaaaagcatacgcatacacatacacacatagagcaagcaagcaggctagcaaccaggaaaggctgccagtgactgctactgggtgtctaagaaccgtagggcggattattgttgcggtggttggttgcgggtggttatgcgatggtacggtgcagaatcgtacggtgttggttatggaattagtatgggtatgtgatatgtggtaatatgtgatattgggttattgtgatttggaatactgaatatcgaatatgggatatggaatatggccatggcatggtatggtatgggatgggagtattctattttattttattttattctggttcctgcgtttagggtagggtaggaagaaggtgagtgcttttgtatataagtggagtgtctggatcagttttgtggattgtgaatgttagtttcccctttaatgtatatttgtattatttgcttttgagtactcaataaccaagcacaactactagttttaaaggatccatcctcttaaacagtacaaatcgcaaagaaaagctccacacccaaaccaaataattgcaagatcttag

SEQ ID NO: 31 - DNA, 28 bp, Artificial Sequence
  Synthetic forward primer KmPDC1_3D_3F_Xba
gcatctagaagagggagaggataaagag

SEQ ID NO: 32 - DNA, 18 bp, Artificial Sequence
  Synthetic reverse primer KmPDC1_3D_4B
tcgtcttctcagctgcaa

SEQ ID NO: 33 - DNA, 1030 bp, kluyveromyces marxianus
gcatctagaagagggagaggataaagagataaattacgattttggattttaatgattttataaacaacaacaaccaaccagccttttactttatttggcatatacacaagcttactccatttcattgattatctatgtgtatatatataagtgatgtataacaattattattatacatagataatatttttatgatatgttttttctgagttttgatattatttattacaagttacaagttacaagttacaagttaccaggaagaattaaataaaggtaaattgggggaattatgagcgtatgggcatagatatatatatattatagagaacaataagctaggggcaatagggaattaattacacaatccatccgaagatcttttcataataccatttcatgtcttttccatgggccttgatataagcggctctttcctcgtctagcaatttgccttcttcttcctccaaatcgactggttctttgaaggagacgagatcgacttcttcggatgggatgagtttggtcttgttccacaacttatacccaacatacgagatgatgtacactgggataccgatgtagcccgtgatgaacgacttgtagtcgaacttatcgccaaggaaggcggtaaagtttttgatcaagccgatcaagcagcagaaaaagagggcgaaccatgcgctgtatggctggaatggggacttgtacgtgagcgcatccttgcttacgccttggactctgaatgctctgtcgaatctgatgtatattatgaggattgagatccaactcatgagtccgaagatggagacgcaattgacgaaatagttgaagacctgtgcgcttccagaggagacgttcatgtaggcgagcaaggcgaacaggattcccatgagcatggaccagtatgggacaccgtttttgtttgttttggcgaagatcttgggggctttgttgtcgatagccaagccgtagagggatcttgaagcgacgtacaagtcggagtttgcagctgagaagacgagagctc

SEQ ID NO: 34 - DNA, 5895 bp, kluyveromyces marxianus
tcgagccgaaagatcgtccgattatccagcgaatatacagcgtgaataatggaatggccttgtattcgttttttccgagagaaaaaaacgggcttcggtgaaaatcgggtgaatatgcaactagcgggacgaatgctctggaaatgcatatcctatgcaactagcgggatgaacaaatctcaccccagaattcgcaggaaaaaacaggaaaaaaaaaaagaaggccaccacggccacaaagaccacaaagaccacaaaaaaaaacaaaaaacaaccgtcccagcttccagtgtttggaatactggaacacaggaagccgcataagagtgggcgttgcacaggaagccaggcccagaagccccagagttacttttttttttttgttttttccttctgttcgctgtgcccgcatcagatgatgcgcctttatttacgatgccaatgcgaatagcaccagtgagagcaccagtaaaagcatacgcatacacatacacacatagagcaagcaagcaggctagcaaccaggaaaggctgccagtgactgctactgggtgtctaagaaccgtagggcggattattgttgcggtggttggttgcgggtggttatgcgatggtacggtgcagaatcgtacggtgttggttatggaattagtatgggtatgtgatatgtggtaatatgtgatattgggttattgtgatttggaatactgaatatcgaatatgggatatggaatatggccatggcatggtatggtatgggatgggagtattctattttattttattttattctggttcctgcgtttagggtagggtaggaagaaggtgagtgcttttgtatataagtggagtgtctggatcagttttgtggattgtgaatgttagtttcccctttaatgtatatttgtattatttgcttttgagtactcaataaccaagcacaactactagttttaaaggatccatcctcttaaacagtacaaatcgcaaagaaaagctccacacccaaaccaaataattgcaagatcttccagtggtgcatgaacgcatgagaaagcccccggaagatcatcttccgggggctttttttttggcgcgcgatacagaccggttcagacaggataaagaggaacgcagaatgttagacaacacccgcttacgcatagctattcagaaatcaggccgtttaagcgatgattcacgagaattgctggcccgctgcggcataaaaattaatttacacactcagcgcctgattgcgatggcggaaaacatgccgattgatatcctgcgcgtgcgtgatgatgacattccgggtctggtaatggatggcgtggtcgatctcggtattatcggcgaaaacgtgctggaagaagagctactcaaccgccgcgcacagggcgaagatccacgctatttaaccctgcgccgtcttgacttcggcggctgccgtttatcgctggcaacaccggttgacgaagcctgggacggcccggccgcgctggacggtaaacgtatcgctacctcatatccgcacctcctcaaacgctacctcgaccagaaaggcgtctcttttaaatcgtgtctgttaaatggttctgtcgaagtcgcgccgcgcgcggggctggccgacgctatctgcgatttggtctctaccggcgcgacgcttgaagctaacggcctgcgtgaagtcgaagttatctaccgctctaaagcctgtctgattcagcgcgacggtgagatggcacagagcaagcaagagctgatcgataaattgctgacccgtattcagggcgtgattcaggcgcgcgaatcgaaatacatcatgatgcacgcgccaagtgaacgcctggaagaggttatcgccctgctgccaggcgccgaaaggccgacaattctgccgctggcaggcgagcaacagcgcgtggcgatgcacatggtcagcagcgaaacgttgttctgggaaaccatggagaaactgaaagcgcttggcgccagctcgattctggtactgccgatcgagaagatgatggagtgatctgacgcctgatggcgctgcgcttatcaggcctacgtaatgcgttgatattttgggttctgtaggccggataaggcggaaccctgtgatggagtaaagaccatgagcttcaataccctgattgactggaacagggatctgaattaattctcatgtttgacagcttatcatcgataagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcttccagtggtgcatgaacgcatgagaaagcccccggaagatcatcttccgggggctttttttttggcgcgcgatacagaccggttcagacaggataaagaggaacgcagaatgttagacaacacccgcttacgcatagctattcagaaatcaggccgtttaagcgatgattcacgagaattgctggcccgctgcggcataaaaattaatttacacactcagcgcctgattgcgatggcggaaaacatgccgattgatatcctgcgcgtgcgtgatgatgacattccgggtctggtaatggatggcgtggtcgatctcggtattatcggcgaaaacgtgctggaagaagagctactcaaccgccgcgcacagggcgaagatccacgctatttaaccctgcgccgtcttgacttcggcggctgccgtttatcgctggcaacaccggttgacgaagcctgggacggcccggccgcgctggacggtaaacgtatcgctacctcatatccgcacctcctcaaacgctacctcgaccagaaaggcgtctcttttaaatcgtgtctgttaaatggttctgtcgaagtcgcgccgcgcgcggggctggccgacgctatctgcgatttggtctctaccggcgcgacgcttgaagctaacggcctgcgtgaagtcgaagttatctaccgctctaaagcctgtctgattcagcgcgacggtgagatggcacagagcaagcaagagctgatcgataaattgctgacccgtattcagggcgtgattcaggcgcgcgaatcgaaatacatcatgatgcacgcgccaagtgaacgcctggaagaggttatcgccctgctgccaggcgccgaaaggccgacaattctgccgctggcaggcgagcaacagcgcgtggcgatgcacatggtcagcagcgaaacgttgttctgggaaaccatggagaaactgaaagcgcttggcgccagctcgattctggtactgccgatcgagaagatgatggagtgatctgacgcctgatggcgctgcgcttatcaggcctacgtaatgcgttgatattttgggttctgtaggccggataaggcggaaccctgtgatggagtaaagaccatgagcttcaataccctgattgactggaacagggatccactagttctagaagagggagaggataaagagataaattacgattttggattttaatgattttataaacaacaacaaccaaccagccttttactttatttggcatatacacaagcttactccatttcattgattatctatgtgtatatatataagtgatgtataacaattattattatacatagataatatttttatgatatgttttttctgagttttgatattatttattacaagttacaagttacaagttacaagttaccaggaagaattaaataaaggtaaattgggggaattatgagcgtatgggcatagatatatatatattatagagaacaataagctaggggcaatagggaattaattacacaatccatccgaagatcttttcataataccatttcatgtcttttccatgggccttgatataagcggctctttcctcgtctagcaatttgccttcttcttcctccaaatcgactggttctttgaaggagacgagatcgacttcttcggatgggatgagtttggtcttgttccacaacttatacccaacatacgagatgatgtacactgggataccgatgtagcccgtgatgaacgacttgtagtcgaacttatcgccaaggaaggcggtaaagtttttgatcaagccgatcaagcagcagaaaaagagggcgaaccatgcgctgtatggctggaatggggacttgtacgtgagcgcatccttgcttacgccttggactctgaatgctctgtcgaatctgatgtatattatgaggattgagatccaactcatgagtccgaagatggagacgcaattgacgaaatagttgaagacctgtgcgcttccagaggagacgttcatgtaggcgagcaaggcgaacaggattcccatgagcatggaccagtatgggacaccgtttttgtttgttttggcgaagatcttgggggctttgttgtcgatagccaagccgtagagggatcttgaagcgacgtacaagtcggagtttgcag

SEQ ID NO: 35 - DNA, 18 bp, Artificial Sequence
  Synthetic forward primer Iden KmPDC1 1F 5D
caccacacacttacccgc

SEQ ID NO: 36 - DNA, 19 bp, Artificial Sequence
  Synthetic forward primer Iden KmPDC1 2B 5D
ggtttggacttcgacttgc

SEQ ID NO: 37 - DNA, 17 bp, Artificial Sequence
  Synthetic forward primer Iden KmPDC1 3F 3D
ccatcaacgccaagcaa

SEQ ID NO: 38 - DNA, 18 bp, Artificial Sequence
  Synthetic forward primer Iden KmPDC1 4B 3D
caaagccggtatcccagt

SEQ ID NO: 39 - DNA, 23 bp, Artificial Sequence
  Synthetic forward primer Iden KmPDC1 1F
atgtctgaaattactctaggtcg

SEQ ID NO: 40 - DNA, 20 bp, Artificial Sequence
  Synthetic forward primer Iden KmPDC1 2B
ttcttgcttggcgttgatgg

SEQ ID NO: 41 - DNA, 29 bp, Artificial Sequence
  Synthetic forward primer Km05Leu2-5UTR_1F_47
catgctcgagatgatgccgtaatcaacac

SEQ ID NO: 42 - DNA, 28 bp, Artificial Sequence
  Synthetic reverse primer Km05Leu2-5UTR_2B_46
gtacagatcttgatgccaagacatttgc

SEQ ID NO: 43 - DNA, 1016 bp, kluyveromyces marxianus
catgctcgagatgatgccgtaatcaacacaaaaactcttccatatttacaacggcccgcttttttcttcttccctttctcacgtcttcaagagcgcccgtaattgatactagaccggttccgggcctgactacatcagttttttgaaaaaaaaaaaaacccactagctagttatacctgattttaagttgagaagagtcatatagccaaagaaccgtttacatgaccttcgtgaagtgccttgatgatcataaaggcatttctatctcttctttttaatataaatatacaaatattgatttcacttcaagaccataacttttaaaccgaacaatttgtatccttcttaactttaagggaaacacattagcagtccaaaagccctaatatactacactccatcaaccatataataaaacatgtcaaagaatatcgttgtcttaccaggtgatcatgtcggtactgaaattaccaatgaagcgatcaaagttttgaacgctatttcagaagcacgcccttctatcaagtttaattttgaacatcatttgattggtggtgctgcaattgatgctactggtgtgccattgccagacgaagctttggaagcttccaagaaagctgatgcagtattattgggtgcagttggtggtccaaagtggggtacaggttccgttagacctgaacaaggtttattgaagatcagaaaggaattgggattatacgctaacttgaggccttgtaattttgcttcagactctttgcttgatctatctccattgaaacctgagtacgccaagggaactgactttgttgtcgttagagaattggtgggtggtatctactttggtgagaggaaggaagatgaaggtgatggtgttgcttgggattctgaaaagtacagtgtaccagaagttcaaagaatcaccagaatggctgcatttttagctttacaacacaacccacctttaccaatctggtcattggataaagcaaatgtcttggcatcaagatctgtac

SEQ ID NO: 44 - DNA, 30 bp, Artificial Sequence
  Synthetic forward primer Km05Leu2-3UTR_3F_48
gtacactagtggctttgactctaagattac

SEQ ID NO: 45 - DNA, 28 bp, Artificial Sequence
  Synthetic reverse primer Km05Leu2_3UTR_4B_48
tgcagagctcgtgaagctgctaggctaa

SEQ ID NO: 46 - DNA, 1011 bp, kluyveromyces marxianus
gtacactagtggctttgactctaagattacacccttttattacgaaataattcaatgctgatacgcccttttgatgtttagcttatatacaatgcacataaactaggttccggttatcctaagatcatgtcatatatatatatatatatatatctttaaacaatcactctcaaaaagtaacattgcagataagcacaaattaatagagaagatatctgtggacataaacaggaatataatataacattttacaaaatcgttattaagcgtatgcatatcttatatcgagaatatagaagggtggatagccaatttaaacgattgctaatgcatgaacaagcgatcgcataccctctattatttcaaaagataacatgactaaactgaatcatgagatgtttagttgtaaggggaatataatcgaaaagggggttgttcaaggcagagaaaggcttccgtcactgaacgagagaaaaaaaacaacaacaaacaagtaacaaaaaaaaaaaaacagagtatttagaagctctgtttatggtttttttaatgaccagaccattcaatagagttcaagtcaataccttctttcaccatttcccacaatggagaaagttctctcaaaaattctactctttctgtaattgccttgatcacatagtcaatttcttcctcagttgtgaatctaccaataccaaatctaatggacgagtgagctagtgcatcatctttacccaatgcatgcaaaacgtaagatggttccaatgatgcagaagtacatgcggagccagaacttaaagcgatgtctcttagagccattagtaatgattcaccttcaacgtatgcaaaagaaatattaacacagcctgggtaacgatggtcaggtgaaccgtttaagatagtatggtcgatagacagtaggcccttcattagtttgtcagatagtctcttgatgtgagcggaatcattgtcatattccttcttcattagcctagcagcttcacgagctctgca

SEQ ID NO: 47 - DNA, 5860 bp, kluyveromyces marxianus
tcgagatgatgccgtaatcaacacaaaaactcttccatatttacaacggcccgcttttttcttcttccctttctcacgtcttcaagagcgcccgtaattgatactagaccggttccgggcctgactacatcagttttttgaaaaaaaaaaaaacccactagctagttatacctgattttaagttgagaagagtcatatagccaaagaaccgtttacatgaccttcgtgaagtgccttgatgatcataaaggcatttctatctcttctttttaatataaatatacaaatattgatttcacttcaagaccataacttttaaaccgaacaatttgtatccttcttaactttaagggaaacacattagcagtccaaaagccctaatatactacactccatcaaccatataataaaacatgtcaaagaatatcgttgtcttaccaggtgatcatgtcggtactgaaattaccaatgaagcgatcaaagttttgaacgctatttcagaagcacgcccttctatcaagtttaattttgaacatcatttgattggtggtgctgcaattgatgctactggtgtgccattgccagacgaagctttggaagcttccaagaaagctgatgcagtattattgggtgcagttggtggtccaaagtggggtacaggttccgttagacctgaacaaggtttattgaagatcagaaaggaattgggattatacgctaacttgaggccttgtaattttgcttcagactctttgcttgatctatctccattgaaacctgagtacgccaagggaactgactttgttgtcgttagagaattggtgggtggtatctactttggtgagaggaaggaagatgaaggtgatggtgttgcttgggattctgaaaagtacagtgtaccagaagttcaaagaatcaccagaatggctgcatttttagctttacaacacaacccacctttaccaatctggtcattggataaagcaaatgtcttggcatcaagatcttccagtggtgcatgaacgcatgagaaagcccccggaagatcatcttccgggggctttttttttggcgcgcgatacagaccggttcagacaggataaagaggaacgcagaatgttagacaacacccgcttacgcatagctattcagaaatcaggccgtttaagcgatgattcacgagaattgctggcccgctgcggcataaaaattaatttacacactcagcgcctgattgcgatggcggaaaacatgccgattgatatcctgcgcgtgcgtgatgatgacattccgggtctggtaatggatggcgtggtcgatctcggtattatcggcgaaaacgtgctggaagaagagctactcaaccgccgcgcacagggcgaagatccacgctatttaaccctgcgccgtcttgacttcggcggctgccgtttatcgctggcaacaccggttgacgaagcctgggacggcccggccgcgctggacggtaaacgtatcgctacctcatatccgcacctcctcaaacgctacctcgaccagaaaggcgtctcttttaaatcgtgtctgttaaatggttctgtcgaagtcgcgccgcgcgcggggctggccgacgctatctgcgatttggtctctaccggcgcgacgcttgaagctaacggcctgcgtgaagtcgaagttatctaccgctctaaagcctgtctgattcagcgcgacggtgagatggcacagagcaagcaagagctgatcgataaattgctgacccgtattcagggcgtgattcaggcgcgcgaatcgaaatacatcatgatgcacgcgccaagtgaacgcctggaagaggttatcgccctgctgccaggcgccgaaaggccgacaattctgccgctggcaggcgagcaacagcgcgtggcgatgcacatggtcagcagcgaaacgttgttctgggaaaccatggagaaactgaaagcgcttggcgccagctcgattctggtactgccgatcgagaagatgatggagtgatctgacgcctgatggcgctgcgcttatcaggcctacgtaatgcgttgatattttgggttctgtaggccggataaggcggaaccctgtgatggagtaaagaccatgagcttcaataccctgattgactggaacagggatctgaattaattctcatgtttgacagcttatcatcgataagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcttccagtggtgcatgaacgcatgagaaagcccccggaagatcatcttccgggggctttttttttggcgcgcgatacagaccggttcagacaggataaagaggaacgcagaatgttagacaacacccgcttacgcatagctattcagaaatcaggccgtttaagcgatgattcacgagaattgctggcccgctgcggcataaaaattaatttacacactcagcgcctgattgcgatggcggaaaacatgccgattgatatcctgcgcgtgcgtgatgatgacattccgggtctggtaatggatggcgtggtcgatctcggtattatcggcgaaaacgtgctggaagaagagctactcaaccgccgcgcacagggcgaagatccacgctatttaaccctgcgccgtcttgacttcggcggctgccgtttatcgctggcaacaccggttgacgaagcctgggacggcccggccgcgctggacggtaaacgtatcgctacctcatatccgcacctcctcaaacgctacctcgaccagaaaggcgtctcttttaaatcgtgtctgttaaatggttctgtcgaagtcgcgccgcgcgcggggctggccgacgctatctgcgatttggtctctaccggcgcgacgcttgaagctaacggcctgcgtgaagtcgaagttatctaccgctctaaagcctgtctgattcagcgcgacggtgagatggcacagagcaagcaagagctgatcgataaattgctgacccgtattcagggcgtgattcaggcgcgcgaatcgaaatacatcatgatgcacgcgccaagtgaacgcctggaagaggttatcgccctgctgccaggcgccgaaaggccgacaattctgccgctggcaggcgagcaacagcgcgtggcgatgcacatggtcagcagcgaaacgttgttctgggaaaccatggagaaactgaaagcgcttggcgccagctcgattctggtactgccgatcgagaagatgatggagtgatctgacgcctgatggcgctgcgcttatcaggcctacgtaatgcgttgatattttgggttctgtaggccggataaggcggaaccctgtgatggagtaaagaccatgagcttcaataccctgattgactggaacagggatccactagtggctttgactctaagattacacccttttattacgaaataattcaatgctgatacgcccttttgatgtttagcttatatacaatgcacataaactaggttccggttatcctaagatcatgtcatatatatatatatatatatatctttaaacaatcactctcaaaaagtaacattgcagataagcacaaattaatagagaagatatctgtggacataaacaggaatataatataacattttacaaaatcgttattaagcgtatgcatatcttatatcgagaatatagaagggtggatagccaatttaaacgattgctaatgcatgaacaagcgatcgcataccctctattatttcaaaagataacatgactaaactgaatcatgagatgtttagttgtaaggggaatataatcgaaaagggggttgttcaaggcagagaaaggcttccgtcactgaacgagagaaaaaaaacaacaacaaacaagtaacaaaaaaaaaaaaacagagtatttagaagctctgtttatggtttttttaatgaccagaccattcaatagagttcaagtcaataccttctttcaccatttcccacaatggagaaagttctctcaaaaattctactctttctgtaattgccttgatcacatagtcaatttcttcctcagttgtgaatctaccaataccaaatctaatggacgagtgagctagtgcatcatctttacccaatgcatgcaaaacgtaagatggttccaatgatgcagaagtacatgcggagccagaacttaaagcgatgtctcttagagccattagtaatgattcaccttcaacgtatgcaaaagaaatattaacacagcctgggtaacgatggtcaggtgaaccgtttaagatagtatggtcgatagacagtaggcccttcattagtttgtcagatagtctcttgatgtgagcggaatcattgtcatattccttcttcattagcctagcagcttcacgagct

SEQ ID NO: 48 - DNA, 28 bp, Artificial Sequence
  Synthetic forward primer Km05PDC5D_1F_50
cgtactcgagccggtgagtcaaagatcg

SEQ ID NO: 49 - DNA, 28 bp, Artificial Sequence
  Synthetic reverse primer Km05PDC5D_2B_50
gatcgaattcagctgataccccaccctt

SEQ ID NO: 50 - DNA, 1028 bp, kluyveromyces marxianus
cgtactcgagccggtgagtcaaagatcgaccagttggtcgaaatcatcaaggttcttggcaccccaacaagagacgagatctgtgccatgaacgaaaactactcagaacacaagttcccacagatcagacccatcccattaaacaagatcttcaagaaagaaacccaggaaacaatagacttgttgtattacatcatgaagtatgatcccaacatcagatacagtgctctccagtgcatgttcaattcagagtacttttcggacatactcgacccacagaaatcaaacgcttcgctaattgagtctctacagcttctagactttgaagaaaacgagttggctggcctgacggccgaagacttggcaaggtttggcgcaaaagtaataataccatcaagctgatcgtgcaagaaggaaaaaaaaaaaaaaaaaaaaaacacaattcatacggtgaagaggagaaaaagaagacgacgacaactgcgaagaagagtaaagaatcatccatccatccattcatacgcacacacgcattctaataatagtattaataataccttcagtaatagtaacattccataggttacgaaaaacttatcacactttacctaacccctctcttaatttaatcatcacgttatgggaaaatagcaatatatatatatatagtatagtatagtatagtatagtatcatttagtataatttagtatagtatagtatagtataatgacttgaaatactacttactactcattcatactaccttgccgtgcctctagcccatgcaggtctcattttttttggttttttcacaggttttagtctacctgttgaaaaaaggaaaaaaaggcgaacagagtagagatccacatttgctgaaaaatatgtggacttaagtattagactcatcatattgtagtctctgcataaaaagtttctgtattttatatatagccacgcttcattatttggaagttattcttgttttgaggctgaataagggtggggtatcagctgaattcgatc

SEQ ID NO: 51 - DNA, 28 bp, Artificial Sequence
  Synthetic forward primer Km05PDC5D_3F_46
cgatactagtgaacttactcctcaaagc

SEQ ID NO: 52 - DNA, 28 bp, Artificial Sequence
  Synthetic reverse primer Km05PDC5D_4B_46
cagtgagctcatcgtgctatcttccatg

SEQ ID NO: 53 - DNA, 1072 bp, kluyveromyces marxianus
cgatactagtgaacttactcctcaaagcaaaagagtaagactttagtttttaaaatttaaattggactttaatagaataatataaggaatatacatcaatgtatggtatatttagtatacttttatattatagatgggcattccctaccctatcacacttaatttattttttcatttatttatttttaaatattgataaacaacactgacactttaatcatgaatgaatatggacaaccttctttactagaatttggaatttagtcgttaatgaaaaaaaaaaaaaagaataaagtatgatgaaaaatataaaggttcaagttgcaaaaaaaaaacaaatgatttcgcccaggatcgaactggggacgttctgcgtgttaagcagatgccataaccgactagaccacgaaaccaacttgttgaaaacaacattttaaaacaggaatatgttcaactattgcagtaggcttttgcccggcacagttccgtacaagtcgaatgattttcactgctttttccgcttttaccttgcccccttgatagatttgggtataaaaggaatgtgggaacactcggcggcaaaaaggtttaggtcaaagtttcttacaaattgctgttttaaatgaatattttaatactatatcgcttatatacatggtcaagttgaatacaatgagtaaattagatgcattgtacttgagtaacgatgattttagtattgtctctgaatggaattgtgagattttccttcaaaatctaagtatacttcatgaagacatgattgttattccttcttcgaacccctatagcaaggtaagctgattttaatctagattctggcgttcgtttcgaaatacactatctatttaactgaaaaaaaattcaatgtactaactggaattggcgcttattccgacgtgtatactctgctaagattatgatcaatattgatggtttagatgagcaaatactaaacgatgtttatgggaaattctgtcttgggagagcattttgggaacgattacagtttgaacctatgtttagcatggaagatagcacgatgagctcactg

SEQ ID NO: 54 - DNA, 5939 bp, kluyveromyces marxianus
tcgagccggtgagtcaaagatcgaccagttggtcgaaatcatcaaggttcttggcaccccaacaagagacgagatctgtgccatgaacgaaaactactcagaacacaagttcccacagatcagacccatcccattaaacaagatcttcaagaaagaaacccaggaaacaatagacttgttgtattacatcatgaagtatgatcccaacatcagatacagtgctctccagtgcatgttcaattcagagtacttttcggacatactcgacccacagaaatcaaacgcttcgctaattgagtctctacagcttctagactttgaagaaaacgagttggctggcctgacggccgaagacttggcaaggtttggcgcaaaagtaataataccatcaagctgatcgtgcaagaaggaaaaaaaaaaaaaaaaaaaaaacacaattcatacggtgaagaggagaaaaagaagacgacgacaactgcgaagaagagtaaagaatcatccatccatccattcatacgcacacacgcattctaataatagtattaataataccttcagtaatagtaacattccataggttacgaaaaacttatcacactttacctaacccctctcttaatttaatcatcacgttatgggaaaatagcaatatatatatatatagtatagtatagtatagtatagtatcatttagtataatttagtatagtatagtatagtataatgacttgaaatactacttactactcattcatactaccttgccgtgcctctagcccatgcaggtctcattttttttggttttttcacaggttttagtctacctgttgaaaaaaggaaaaaaaggcgaacagagtagagatccacatttgctgaaaaatatgtggacttaagtattagactcatcatattgtagtctctgcataaaaagtttctgtattttatatatagccacgcttcattatttggaagttattcttgttttgaggctgaataagggtggggtatcagctgaattcagatcttccagtggtgcatgaacgcatgagaaagcccccggaagatcatcttccgggggctttttttttggcgcgcgatacagaccggttcagacaggataaagaggaacgcagaatgttagacaacacccgcttacgcatagctattcagaaatcaggccgtttaagcgatgattcacgagaattgctggcccgctgcggcataaaaattaatttacacactcagcgcctgattgcgatggcggaaaacatgccgattgatatcctgcgcgtgcgtgatgatgacattccgggtctggtaatggatggcgtggtcgatctcggtattatcggcgaaaacgtgctggaagaagagctactcaaccgccgcgcacagggcgaagatccacgctatttaaccctgcgccgtcttgacttcggcggctgccgtttatcgctggcaacaccggttgacgaagcctgggacggcccggccgcgctggacggtaaacgtatcgctacctcatatccgcacctcctcaaacgctacctcgaccagaaaggcgtctcttttaaatcgtgtctgttaaatggttctgtcgaagtcgcgccgcgcgcggggctggccgacgctatctgcgatttggtctctaccggcgcgacgcttgaagctaacggcctgcgtgaagtcgaagttatctaccgctctaaagcctgtctgattcagcgcgacggtgagatggcacagagcaagcaagagctgatcgataaattgctgacccgtattcagggcgtgattcaggcgcgcgaatcgaaatacatcatgatgcacgcgccaagtgaacgcctggaagaggttatcgccctgctgccaggcgccgaaaggccgacaattctgccgctggcaggcgagcaacagcgcgtggcgatgcacatggtcagcagcgaaacgttgttctgggaaaccatggagaaactgaaagcgcttggcgccagctcgattctggtactgccgatcgagaagatgatggagtgatctgacgcctgatggcgctgcgcttatcaggcctacgtaatgcgttgatattttgggttctgtaggccggataaggcggaaccctgtgatggagtaaagaccatgagcttcaataccctgattgactggaacagggatctgaattaattctcatgtttgacagcttatcatcgataagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcttccagtggtgcatgaacgcatgagaaagcccccggaagatcatcttccgggggctttttttttggcgcgcgatacagaccggttcagacaggataaagaggaacgcagaatgttagacaacacccgcttacgcatagctattcagaaatcaggccgtttaagcgatgattcacgagaattgctggcccgctgcggcataaaaattaatttacacactcagcgcctgattgcgatggcggaaaacatgccgattgatatcctgcgcgtgcgtgatgatgacattccgggtctggtaatggatggcgtggtcgatctcggtattatcggcgaaaacgtgctggaagaagagctactcaaccgccgcgcacagggcgaagatccacgctatttaaccctgcgccgtcttgacttcggcggctgccgtttatcgctggcaacaccggttgacgaagcctgggacggcccggccgcgctggacggtaaacgtatcgctacctcatatccgcacctcctcaaacgctacctcgaccagaaaggcgtctcttttaaatcgtgtctgttaaatggttctgtcgaagtcgcgccgcgcgcggggctggccgacgctatctgcgatttggtctctaccggcgcgacgcttgaagctaacggcctgcgtgaagtcgaagttatctaccgctctaaagcctgtctgattcagcgcgacggtgagatggcacagagcaagcaagagctgatcgataaattgctgacccgtattcagggcgtgattcaggcgcgcgaatcgaaatacatcatgatgcacgcgccaagtgaacgcctggaagaggttatcgccctgctgccaggcgccgaaaggccgacaattctgccgctggcaggcgagcaacagcgcgtggcgatgcacatggtcagcagcgaaacgttgttctgggaaaccatggagaaactgaaagcgcttggcgccagctcgattctggtactgccgatcgagaagatgatggagtgatctgacgcctgatggcgctgcgcttatcaggcctacgtaatgcgttgatattttgggttctgtaggccggataaggcggaaccctgtgatggagtaaagaccatgagcttcaataccctgattgactggaacagggatccactagtgaacttactcctcaaagcaaaagagtaagactttagtttttaaaatttaaattggactttaatagaataatataaggaatatacatcaatgtatggtatatttagtatacttttatattatagatgggcattccctaccctatcacacttaatttattttttcatttatttatttttaaatattgataaacaacactgacactttaatcatgaatgaatatggacaaccttctttactagaatttggaatttagtcgttaatgaaaaaaaaaaaaaagaataaagtatgatgaaaaatataaaggttcaagttgcaaaaaaaaaacaaatgatttcgcccaggatcgaactggggacgttctgcgtgttaagcagatgccataaccgactagaccacgaaaccaacttgttgaaaacaacattttaaaacaggaatatgttcaactattgcagtaggcttttgcccggcacagttccgtacaagtcgaatgattttcactgctttttccgcttttaccttgcccccttgatagatttgggtataaaaggaatgtgggaacactcggcggcaaaaaggtttaggtcaaagtttcttacaaattgctgttttaaatgaatattttaatactatatcgcttatatacatggtcaagttgaatacaatgagtaaattagatgcattgtacttgagtaacgatgattttagtattgtctctgaatggaattgtgagattttccttcaaaatctaagtatacttcatgaagacatgattgttattccttcttcgaacccctatagcaaggtaagctgattttaatctagattctggcgttcgtttcgaaatacactatctatttaactgaaaaaaaattcaatgtactaactggaattggcgcttattccgacgtgtatactctgctaagattatgatcaatattgatggtttagatgagcaaatactaaacgatgtttatgggaaattctgtcttgggagagcattttgggaacgattacagtttgaacctatgtttagcatggaagatagcacgatgagct

SEQ ID NO: 55 - DNA, 21 bp, Artificial Sequence
  Synthetic forward primer KmPDC5 RT 1F
ctgtacgccgtcacagttccc

SEQ ID NO: 56 - DNA, 20 bp, Artificial Sequence
  Synthetic reverse primer KmPDC5 RT 2B
aagccgatcgcggatgtacc

SEQ ID NO: 57 - DNA, 18 bp, Artificial Sequence
  Synthetic forward primer KmPDC5in_1F
tcttataccgctcgcaga

SEQ ID NO: 58 - DNA, 18 bp, Artificial Sequence
  Synthetic reverse primer KmPDC5in_2B
aggagaacaggaacatcc

SEQ ID NO: 59 - DNA, 18 bp, Artificial Sequence
  Synthetic forward primer KmPDC5-1373_1F
atgcccatgtcgctatac

SEQ ID NO: 60 - DNA, 19 bp, Artificial Sequence
  Synthetic reverse primer ScURA3_N_2B
gcagcacgttccttatatg

SEQ ID NO: 61 - DNA, 6800 bp, Artificial Sequence
  Synthetic pKI vector
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcagatcttccagtggtgcatgaacgcatgagaaagcccccggaagatcatcttccgggggctttttttttggcgcgcgatacagaccggttcagacaggataaagaggaacgcagaatgttagacaacacccgcttacgcatagctattcagaaatcaggccgtttaagcgatgattcacgagaattgctggcccgctgcggcataaaaattaatttacacactcagcgcctgattgcgatggcggaaaacatgccgattgatatcctgcgcgtgcgtgatgatgacattccgggtctggtaatggatggcgtggtcgatctcggtattatcggcgaaaacgtgctggaagaagagctactcaaccgccgcgcacagggcgaagatccacgctatttaaccctgcgccgtcttgacttcggcggctgccgtttatcgctggcaacaccggttgacgaagcctgggacggcccggccgcgctggacggtaaacgtatcgctacctcatatccgcacctcctcaaacgctacctcgaccagaaaggcgtctcttttaaatcgtgtctgttaaatggttctgtcgaagtcgcgccgcgcgcggggctggccgacgctatctgcgatttggtctctaccggcgcgacgcttgaagctaacggcctgcgtgaagtcgaagttatctaccgctctaaagcctgtctgattcagcgcgacggtgagatggcacagagcaagcaagagctgatcgataaattgctgacccgtattcagggcgtgattcaggcgcgcgaatcgaaatacatcatgatgcacgcgccaagtgaacgcctggaagaggttatcgccctgctgccaggcgccgaaaggccgacaattctgccgctggcaggcgagcaacagcgcgtggcgatgcacatggtcagcagcgaaacgttgttctgggaaaccatggagaaactgaaagcgcttggcgccagctcgattctggtactgccgatcgagaagatgatggagtgatctgacgcctgatggcgctgcgcttatcaggcctacgtaatgcgttgatattttgggttctgtaggccggataaggcggaaccctgtgatggagtaaagaccatgagcttcaataccctgattgactggaacagggatctgaattaattctcatgtttgacagcttatcatcgataagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcttccagtggtgcatgaacgcatgagaaagcccccggaagatcatcttccgggggctttttttttggcgcgcgatacagaccggttcagacaggataaagaggaacgcagaatgttagacaacacccgcttacgcatagctattcagaaatcaggccgtttaagcgatgattcacgagaattgctggcccgctgcggcataaaaattaatttacacactcagcgcctgattgcgatggcggaaaacatgccgattgatatcctgcgcgtgcgtgatgatgacattccgggtctggtaatggatggcgtggtcgatctcggtattatcggcgaaaacgtgctggaagaagagctactcaaccgccgcgcacagggcgaagatccacgctatttaaccctgcgccgtcttgacttcggcggctgccgtttatcgctggcaacaccggttgacgaagcctgggacggcccggccgcgctggacggtaaacgtatcgctacctcatatccgcacctcctcaaacgctacctcgaccagaaaggcgtctcttttaaatcgtgtctgttaaatggttctgtcgaagtcgcgccgcgcgcggggctggccgacgctatctgcgatttggtctctaccggcgcgacgcttgaagctaacggcctgcgtgaagtcgaagttatctaccgctctaaagcctgtctgattcagcgcgacggtgagatggcacagagcaagcaagagctgatcgataaattgctgacccgtattcagggcgtgattcaggcgcgcgaatcgaaatacatcatgatgcacgcgccaagtgaacgcctggaagaggttatcgccctgctgccaggcgccgaaaggccgacaattctgccgctggcaggcgagcaacagcgcgtggcgatgcacatggtcagcagcgaaacgttgttctgggaaaccatggagaaactgaaagcgcttggcgccagctcgattctggtactgccgatcgagaagatgatggagtgatctgacgcctgatggcgctgcgcttatcaggcctacgtaatgcgttgatattttgggttctgtaggccggataaggcggaaccctgtgatggagtaaagaccatgagcttcaataccctgattgactggaacagggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

(19) United States
(12) Patent Application Publication
(10) Pub. No.: US 2015/0225733 A1
(43) Pub. Date: Aug. 13, 2015

(54) YEAST CELL HAVING ENHANCED GENETIC MANIPULATION EFFICIENCY AND USE THEREOF

(71) Applicants: Samsung Electronics Co., Ltd., Suwon-si (KR); Chung-Ang University Industry-Academy Cooperation Foundation, Seoul (KR)

(72) Inventors: Jae-young Kim, Suwon-si (KR); Changduk Kang, Gwacheon-si (KR); Hyunah Kang, Seoul (KR); Jinho Choo, Seoul (KR); Changpyo Han, Bucheon-si (KR); Kwangmyung Cho, Seongnam-si (KR)

(21) Appl. No.: 14/622,182

(22) Filed: Feb. 13, 2015

(30) Foreign Application Priority Data
Feb. 13, 2014 (KR) 10-2014-0016792

(51) Int. Cl. C12N 15/81; C12P 7/24; C12P 7/56; C12P 7/18

(52) U.S. Cl. CPC C12N 15/815; C12P 7/18; C12P 7/24; C12P 7/56

(57) ABSTRACT

A recombinant yeast cell having enhanced genetic manipulation efficiency, a method of preparing the recombinant yeast cell, and a method of preparing a biochemical by using the same.

### RELATED APPLICATION

This application claims the benefit of Korean Patent Application No. 10-2014-0016792, filed on Feb. 13, 2014, in the Korean Intellectual Property Office, the entire disclosure of which is hereby incorporated by reference.

### INCORPORATION BY REFERENCE OF ELECTRONICALLY SUBMITTED MATERIALS

Incorporated by reference in its entirety herein is a computer-readable nucleotide/amino acid sequence listing submitted herewith and identified as follows: One 87,280 byte ASCII (Text) file named “718708_ST25.TXT” created Feb. 11, 2015.

### BACKGROUND

1. Field

The present disclosure relates to methods and apparatuses for yeast cells having enhanced genetic manipulation efficiency and the use thereof.

2. Description of the Related Art

Metabolic engineering refers to a series of experiments and prediction technology that includes introducing a new metabolic pathway or deletion, amplification, or change to the existing metabolic pathway by using a genetic manipulation technology to change metabolic properties of cells or strains in a desirable direction. Such technology may be used to combine components of an organism in various ways to change a system into an efficient system suitable for a purpose or to develop a novel biological system.

A specific gene may be deleted or inserted by using the genetic manipulation technology, such that a target cell may have desired properties. Generally, a method of inserting a DNA fragment, which is to be substituted with a target gene through homologous recombination, into a chromosome for deletion of a specific gene is widely used.

Yeasts are eukaryotes that have various advantages, compared to prokaryotes such as Escherichia coli. Yeasts such as Saccharomyces cerevisiae may be easy to genetically manipulate but some yeasts such as Kluyveromyces marxianus have relatively low genetic manipulation efficiency.

Therefore, a strain having enhanced genetic manipulation efficiency through metabolic engineering and a method of preparing the strain are needed for yeast strains having relatively low genetic manipulation efficiency, such as Kluyveromyces marxianus.

### SUMMARY

Provided is a recombinant yeast cell having enhanced genetic manipulation efficiency, wherein the recombinant yeast cell has a Crabtree-negative phenotype and reduced or eliminated Ku80 polypeptide activity.

Also provided is a method of providing the recombinant yeast cell. In one aspect, the method comprising introducing a mutation in a Ku80 gene of the Crabtree-negative yeast cell, wherein the mutation reduces or eliminates Ku80 polypeptide activity and non-homologous end-joining activity in the yeast cell. In another aspect, the method comprising providing a Ku80 gene deletion cassette, wherein the cassette comprises a gene-specific homologous region that has a sequence identity with a portion of the Ku80 gene sufficient to facilitate homologous recombination of the cassette with the portion of the Ku80 gene; and inserting the cassette into a Crabtree-negative yeast cell, whereby at least a portion of the Ku80 gene is deleted.

Further provided is a method of producing biochemicals using recombinant yeast cell that further comprises an exogenous gene involved in a biochemical pathway.

Additional aspects will be set forth in part in the description which follows and, in part, will be apparent from the description, or may be learned by practice of the presented embodiments.

### BRIEF DESCRIPTION OF THE DRAWINGS

These and/or other aspects will become apparent and more readily appreciated from the following description of the embodiments, taken in conjunction with the accompanying drawings in which:

FIG. 1 is a schematic view of a pKI-KmKU80DsU2 vector including a ku80 gene deletion cassette;

FIG. 2 is a schematic view of PCR1, PCR2, and PCR3 before and after deletion of a ku80 gene;

FIG. 3 is schematic view of PCR amplification regions for confirmation of deletion of URA3;

FIG. 4 is a schematic view of a pKI-Km05PDC1DU53 vector including a pdcl gene deletion cassette;

FIG. 5 is a schematic view of PCR7, PCR8, and PCR9 before and after deletion of a pdc1 gene;

FIG. 6 is a schematic view of a pKI-Km05LEU2DU2 vector including a leu2 gene deletion cassette;

FIG. 7 is a schematic view of a pKI-Km05PDC5DU2 vector including a pdc5 gene deletion cassette; and

FIG. 8 is a schematic view of PCR10 and PCR11 amplification regions before and after deletion of a pdc5 gene, which can be used for confirmation of deletion of the pdc5 gene.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

According to an aspect of the present disclosure, provided are recombinant yeast cells having inactivated or attenuated activities of a Ku80 polypeptide (i.e. the activity of a Ku80 polypeptide has been reduced or eliminated). The term “recombinant” is used herein to refer to a cell or molecule (e.g., protein or nucleic acid) that has been genetically engineered and is non-naturally occurring.

The Ku80 polypeptide participates in repairing a genomic damage caused by a DNA double strand break (DSB). The process of repairing a DSB may occur via homologous recombination (HR) or non-homologous end joining (NHEJ). The Ku80 polypeptide forms a Ku heterodimer with Ku70 and then binds to DNA DSB ends to participate in a non-homologous end coupling path. The Ku80 polypeptide may be an enzyme classified as EC 3.6.1. The Ku80 polypeptide may have an amino acid sequence of SEQ ID NO. 1. A gene for encoding the Ku80 polypeptide may have a nucleotide sequence of SEQ ID NO. 2.

As used herein, the term “activity increase”, “enzyme activity increase”, “increased activity”, or “increased enzyme activity” denotes that a cell, a polypeptide or an isolated enzyme has an increased activity level, compared to an activity level of a comparable cell of the same type or to the original polypeptide or enzyme (polypeptide or enzyme without a given mutation, such as a “wild-type” enzyme). Increased activity encompasses activity (e.g., an enzyme conversion activity from a substrate to a product) that is at least about 5%, at least about 10%, at least about 15%, at least about 20%, at least about 30%, at least about 50%, at least about 60%, at least about 70%, or at least about 100% increased compared to the same biochemical conversion activity of the original (e.g., “wild-type”) cell, polypeptide, or enzyme. Increased activity of a cell, polypeptide, or enzyme may be confirmed by using any method commonly known in the art.

As used herein, “inactivated” or “reduced” activity of a cell, an enzyme or a polypeptide denotes a cell, enzyme, or polypeptide having an activity level that is lower than an activity level measured in a comparable cell of the same type, or the activity level of the original (e.g., “wild-type”) enzyme. “Inactivated” or “reduced” activity a cell, an enzyme or a polypeptide encompasses a cell, enzyme, or polypeptide the activity of which has been eliminated, such that there is no detectable activity. Reduced activity encompasses activity (e.g., an enzyme conversion activity from a substrate to a product) that is reduced by about 10% or more, 20% or more, about 30% or more, about 40% or more, about 50% or more, about 55% or more, about 60% or more, about 70% or more, about 75% or more, about 80% or more, about 85% or more, about 90% or more, about 95% or more, or about 100% as compared to the activity of the original (e.g., “wild-type”) cell, polypeptide, or enzyme. Reduced activity of the cell, polypeptide, or enzyme may be confirmed by using a commonly known method in the art. The inactivation or reduction includes a case in which the polypeptide or enzyme is inactive or has reduced activity even though the enzyme is expressed, and a case in which the level of expression is decreased compared to an unmanipulated gene.

Activity of the enzyme or polypeptide of the yeast cell may be inactivated or reduced due to mutation such as deletion or disruption of a gene that encodes the enzyme or polypeptide. As used herein, the “deletion” or “disruption” of the gene includes the case where a part or a whole gene, or a part or a whole regulatory region of a promoter or terminator of the gene is mutated, substituted, deleted, or at least one base is inserted into the gene when the gene is not expressed or has a reduced amount of expression, or activity of the enzyme is removed or reduced even when the gene is expressed. The deletion or disruption of the gene may be caused by genetic engineering such as homologous recombination, mutation induction, or molecular evolution. When a cell includes a plurality of the same genes or at least two different polypeptide paralogs, at least one gene may be deleted or disrupted.

The enzyme or polypeptide may be inactivated or has reduced activity due to substitution, addition, or deletion of a part of or the entire gene encoding the enzyme or polypeptide. For example, the inactivation or reduced activity of the enzyme or polypeptide may be caused by homologous recombination or may be performed by transforming a vector including some sequence of the gene to the cell, culturing the cell so that the sequence may be homogonously recombined with an endogenous gene of the cell, and then selecting cells, in which the homologous recombination occurred, by using a selection marker.

Inactivation or reduced activity of the Ku80 polypeptide may be due to a cassette for deletion of a ku80 gene (i.e. a ku80 gene deletion cassette), wherein the cassette includes a gene-specific homologous region which is homologous to a portion of the ku80 gene. Also, inactivation or reduced activity of the Ku80 polypeptide may be due to random mutagenesis such as UV irradiation, insertion inactivation, antisense-RNA, or siRNA. As used herein, the term “gene” denotes a nucleic acid (e.g., polynucleotide) expressing a specific protein, which optionally includes a regulatory sequence of a 5′-non-coding sequence and/or 3′-non-coding sequence.

The recombinant yeast cell may have a gene encoding the Ku80 polypeptide that is inactivated or has reduced activity. The term “inactivation” or “inactivated” may denote a gene that is not expressed or a gene that is produced but does not have activity. The term “reduction” or “reduced” as used herein may refer to a recombinant yeast cell that shows a lower level of expression than an unmanipulated yeast cell, or even if expressed, has a low level of activity. The activity of the Ku80 polypeptide may be reduced by about 20% or greater, 30% or greater, 40% or greater, 50% or greater, about 55% or greater, about 60% or greater, about 70% or greater, about 75% or greater, about 80% or greater, about 85% or greater, about 90% or greater, about 95% or greater, or about 100% compared to a suitable control group.

The cassette deleting the ku80 gene (i.e. the ku80 gene deletion cassette) may be a DNA module having a certain structure for deleting the ku80 gene by using a homologous sequence. The term “homologous” as used herein refers to a sequence having sufficient sequence identity to the ku80 gene or a portion thereof to facilitate homologous recombination, for example, a sequence identity of about 90% or greater, 95% or greater, or 99% or greater.

The cassette deleting the ku80 gene may further include a promoter specific homologous region that is homologous to a portion of the promoter of the ku80 or a marker gene that is operably linked to the promoter specific homologous region. The gene specific homologous region, which is homologous to at least some portions of the ku80 gene, may be connected at the downstream of the marker gene.

The promoter specific homologous region may include, for example, a sequence that is identical to a portion of a promoter sequence of the ku80 gene at a sequence identity of 90% or greater, 95% or greater, or 99% or greater. The promoter specific homologous region may be, for example, homologous to about 40 nucleotides (nts) to about 200 nts, 40 nts to about 150 nts, about 40 nts to about 100 nts, or about 40 nts to about 80 nts in a 3′-end to a 5′-end direction of the promoter of the ku80 gene.

The expression “operably linked to promoter-specific homologous region” means an encoding nucleotide sequence (e.g., a marker gene) is connected such that the encoding sequence (e.g., marker gene) may be expressed by a promoter when the promoter specific homologous region is integrated in the promoter of the target gene. For example, when the promoter specific homologous region does not undergo homologous recombination, the marker gene that is operably linked to the promoter specific homologous region may not be expressed. For example, when the promoter specific homologous region homologously recombines with a portion of the promoter of the target gene, the marker gene that is operably linked to the promoter specific homologous region may not be expressed.

The marker gene may be, for example, an antibiotic resistance gene or a fluorescent protein gene. The antibiotic resistance gene may be, for example, selected from the group consisting of an ampicillin resistance gene, a kanamycin resistance gene, a chloramphenicol resistance gene, and a tetracycline gene. The fluorescent protein may be, for example, selected from the group consisting of a yeast-enhanced green fluorescent protein (yEGFP) gene, a green fluorescent protein (GFP) gene, a blue fluorescent protein (BFP) gene, and a red fluorescent protein (RFP). The marker gene may include a transcription terminator. The transcription terminator may be a transcription terminator of an iso-1-cytochrome C gene (CYC1), a transcription terminator of a phosphoribosyl-anthranilate isomerase (TRP1) gene, or a transcription terminator of an alcohol dehydrogenase 1 (ADH1) gene.

The gene specific homologous region homologous to a portion of the Ku80 gene may be homologous to, for example, about 500 nts to about 1500 nts or about 500 nts to about 1000 nts of the Ku80 gene.

The expression “genetic manipulation or genetic engineering” or “genetic recombination” as used herein may include modifications such as insertion of an expressible nucleic acid encoding for a polypeptide, addition of another nucleic acid, and deletion of nucleic acid and/or destruction of other functions of genetic materials of yeast cells. Genetic manipulation may be related to translation and modifications after translation that cause changes in enzymatic activity and/or selectivity, and/or provision of an additional polynucleotide such as a recombinant having an increased copy number of an enzyme related to the production of a polypeptide, under selected culture conditions.

The recombinant yeast cell may have enhanced genetic manipulation efficiency. The expression “enhanced genetic manipulation efficiency” as used herein indicates higher efficiency in the deletion and expression of an endogenous nucleic acid of a recombinant cell or the introduction and expression of an exogenous nucleic acid into a recombinant cell than in a comparable type of cell. For example, the genetic manipulation efficiency may be increased by about 50% or greater, about 100% or greater, about 200% or greater, about 300% or greater, about 400% or greater, about 500% or greater, about 600% or greater, about 700% or greater, about 800% or greater, about 900% or greater, about 1000% or greater, about 1100% or greater, about 1200% or greater, about 1300% or greater, about 1400% or greater, or about 1500% or greater than an unmanipulated cell under a standard condition with respect to deletion or insertion of a desired gene. The enhanced genetic manipulation efficiency may be confirmed by any method known in the art. For example, measuring the enhanced genetic manipulation efficiency for the Kluyveromyces lactis is described in Kooistra et. al, Yeast 2004 21: 781-792. Also, measuring the enhanced genetic manipulation efficiency for the Yarrowia lipolytica is described in Jonathan et. al, Biotechnol Lett 2013 35(4) p571-6.

The recombinant yeast cell may have inactivated or reduced activity of NHEJ and as a result, enhanced genetic manipulation efficiency. Not all yeast cells with inactivated or reduced activity of the Ku80 polypeptide result in enhanced genetic manipulation efficiency through the inactivation or reduced activity of NHEJ. The NHEJ may not operate in all yeast cells. Also, in some yeast cells, such as Yarrowia lipolytica, the yeast cells that were deleted of the ku80 gene did not show enhanced genetic manipulation efficiency, compared to unmanipulated yeast cells in which the ku80 gene was not deleted. In one embodiment of this invention, the Kluyveromyces marxianus having a deletion of the ku80 gene shows enhanced genetic manipulation efficiency, compared to unmanipulated yeast cells in which the ku80 gene was not deleted.

Also, the yeast cell may be a type of ascomycota. The ascomycota may be saccharomycetaceae. The saccharomycetaceae may be Saccharomyces genus, Kluyveromyces genus, Candida genus, Pichia genus, Issatchenkia genus, Debaryomyces genus, Zygosaccharomyces genus, or Saccharomycopsis genus. The Kluyveromyces genus may be kluyveromyces marxianus, kluyveromyces lactis, Kluyveromyces waltii, or Kluyveromyces thermotolerans. The Candida genus may be Candida utilis or Candida glabrata.

Also, the recombinant yeast cell may have a Crabtree-negative phenotype. The Crabtree effect is a phenomenon in which cellular respiration is inhibited when highly concentrated glucose is added to an aerobic culture medium and thus, a yeast cell having a Crabtree-negative phenotype does not exhibit glucose mediated inhibition of oxygen consumption. The yeast cell having a Crabtree-negative phenotype may be Saccharomyces genus, Kluyveromyces genus, Pichia genus, Hansenula genus, Issatchenkia genus, or Candida genus. The yeast cell having a Crabtree-negative phenotype may be Kluyveromyces marxianus, Kluyveromyces lactis, Kluyveromyces waltii, Saccharomyces kluyveri, Pichia anomala, Pichia stipitis, Pichia kudriavzevii, Issatchenkia orientalis, Hansenula anomala, or Candida utilis.

The recombinant yeast cell may be a yeast cell in which a gene that participates in a biochemical synthesis pathway is inserted therein. The biochemical may be an organic acid. The organic acid may be a C1 to C20 organic acid. The organic acid may be acetic acid, lactic acid, propionic acid, 3-hydroxy propionic acid, butyric acid, 4-hydroxybutyric acid, succinic acid, fumaric acid, malic acid, citric acid, oxalic acid, adipic acid, or a combination thereof. In addition, the biochemical may be fumarate, malate, acrylate, 1,4-butanediol, 4-hydroxy-butyraldehyde, 1,2-propanediol, 1,3-propanediol, ethylene glycol, and lactate (see U.S. Pat. No. 8,129,154, U.S. Pat. No. 8,293,951, US20120225463, US 20130189751, US 20090053782, and KR20130001509). The gene that participates in the biochemical biosynthesis pathway is a gene related to a production pathway of the biochemical.

The gene that participates in the biochemical biosynthesis pathway may not only include a gene that produces a precursor of the biochemical but also a gene in a biochemical pathway that has a synergistic or a competitive relationship to the biochemical biosynthesis pathway. The gene may be at least one gene encoding at least one polypeptide selected from the group consisting of succinyl-CoA synthetase (SucCD), a-ketoglutarate decarboxylase (SucA), CoA-dependent succinate semialdehyde dehydrogenase (SucD), 4-hydroxybutyrate dehydrogenase (4Hbd), 4-hydroxybutyryl CoA-transferase (Cat2), butyraldehyde dehydrogenase (Bid), aldehyde/alcohol dehydrogenase (AADH), succinyl-CoA:coenzyme A transferase (Cat1), alcohol dehydrogenase (Adh), lactate dehydrogenase (Ldh), and combination thereof.

Also, the yeast cell may be a natural yeast cell modified to delete or reduce Ku80 activity, or a yeast cell that has been previously or subsequently further engineered or mutated to facilitate production of a desired product, such as the biochemical described above, or to have other desirable characteristics. The mutant yeast cell may further have resistance to, for example, ampicillin, uracil, sulfaguanidine, sulfathiazole, azaserine, trimethoprim, or monofluoroacetate.

According to another aspect of the present disclosure, provided is a method of preparing a yeast cell in which a ku80 gene has been deleted, the method including: preparing a cassette for deleting the ku80 gene (i.e. a ku80 gene deletion cassette), in which the cassette includes a gene specific homologous region that is homologous to a portion of the ku80 gene; inserting the cassette into a yeast cell; and identifying a yeast cell from which the ku80 gene was deleted among yeast cells in which the cassette was inserted.

The cassette deleting the ku80 gene and the yeast cell are as described above.

In the method described above, the cassette inserted into the yeast cell may be integrated into a genome of the yeast cell through a homologous recombination. In other words, as a result of the insertion, a homologous recombination may occur between the promoter specific homologous region of the cassette and a target region thereof, and a gene specific homologous region and a target region thereof, such that the ku80 gene may be deleted.

The preparation process described above may include amplification by using, for example, a polynucleotide including a marker gene as a template, a forward primer including a 5′ terminal sequence of the marker gene and a sequence of a ku80 promoter specific homologous region, and a reverse primer including a 3′ terminal sequence of the marker gene and a sequence of the ku80 gene specific homologous region to obtain amplification products. The template polynucleotide may have, for example, a plasmid for including the marker gene. The marker gene is as described above.

The deletion of the ku80 gene may be, for example, confirmed by a protein that is expressed from the marker gene of the cassette that has been integrated into a chromosome of a host cell. The marker gene may be, for example, an antibiotic resistance gene. The antibiotic resistance gene may be as described above. When the marker gene is an antibiotic resistance gene, the confirmation process may include, for example, confirming proliferation of cells in a medium including an antibiotic. Also, the marker gene may be, for example, a fluorescent protein gene. The fluorescent protein gene is as described above. When the marker gene is a fluorescent protein gene, the confirmation process may involve identifying cells expressing fluorescence.

According to another aspect of the present disclosure, provided is a method of manipulating a desired gene by using a recombinant yeast cell having enhanced genetic manipulation efficiency.

The desired gene includes a gene that is inserted into the yeast cell from the outside environment (e.g., exogenous gene), such as a gene that participates in the production of useful products and/or a gene pre-existing in the yeast cell for genetic manipulation such as deletion. The yeast cell may also enable the efficient deletion of a specific gene in the yeast cell. For example, a gene encoding for an enzyme that converts pyruvate into acetaldehyde, a gene encoding for 3-isopropyl malate, a gene encoding for an enzyme that converts lactate into pyruvate, a gene encoding for an enzyme that converts dihydroxyacetone phosphate (DHAP) into glycerol-3-phosphate, an ndel gene or an nde2 gene encoding for an external mitochondrial NADH dehydrogenase, a gene encoding for an enzyme that converts acetyl-CoA into ethanol, a gene encoding for an enzyme that converts oxaloacetate into malate, or a gene encoding for a factor that controls aerobic respiration in the yeast cell may be efficiently inactivated or reduced. In other words, the yeast cell may have additionally deleted or reduced activity of a polypeptide that converts pyruvate into acetaldehyde, a 3-isopropylmalate dehydrogenase polypeptide, a polypeptide that converts lactate into pyruvate, a polypeptide that converts DHAP into glycerol-3-phosphate, external mitochondrial NADH dehydrogenase activities, a polypeptide that converts acetyl-CoA into ethanol, a polypeptide that converts oxaloacetate into malate, a polypeptide encoding for a factor that controls aerobic respiration, or a combination thereof. Alternatively, the yeast cell may have additionally inactivated or reduced activity of a gene encoding for a polypeptide that converts pyruvate into acetaldehyde, a gene encoding for a 3-isopropyl malate dehydrogenase polypeptide, a gene that converts lactate into pyruvate, a gene that converts DHAP into glycerol-3-phosphate, an ndel gene or an nde2 gene encoding for an external mitochondrial NADH dehydrogenase, a gene that converts acetyl-CoA into ethanol, a gene that converts oxaloacetate into malate, and a gene encoding for a factor that controls aerobic respiration.

The polypeptide that converts pyruvate into acetaldehyde may be an enzyme classified as EC 4.1.1.1. The polypeptide that converts pyruvate into acetaldehyde may have an amino acid sequence of SEQ ID NO. 3 or 5. The gene encoding for a polypeptide that converts pyruvate into acetaldehyde may have a nucleotide sequence of SEQ ID NO. 4 or 6. The gene may be pdc1, pdc2, or pdc5 encoding for a pyruvate decarboxylase (Pdc).

The 3-isopropylmalate dehydrogenase may be an enzyme classified as EC 1.1.1.85. The 3-isopropylmalate dehydrogenase produces 4-methyl-2-oxopentanoate from 3-isopropylmalate, and the product obtained therefrom corresponds to an intermediate in the biosynthesis of leucine. The 3-isopropyl malate dehydrogenase polypeptide may have an amino acid sequence of SEQ ID NO. 7. The gene encoding for the 3-isopropyl malate dehydrogenase may have a nucleotide sequence of SEQ ID NO. 8. The gene may be leu2 encoding for the 3-isopropyl malate dehydrogenase.

The polypeptide that converts lactate into pyruvate may be a cytochrome c-dependent enzyme. The polypeptide that converts lactate into pyruvate may be a lactate cytochrome c-oxidoreductase (CYB2). The CYB2 may be an enzyme classified as EC 1.1.2.4, which acts on D-lactate or as EC 1.1.2.3, which acts on L-lactate.

The polypeptide that converts DHAP into glycerol-3-phosphate may be a cytosolic glycerol-3-phosphate dehydrogenase (GPD1), which is an enzyme that uses oxidation of NADH into NAD+ to catalyze a reduction of DHAP into glycerol-3-phosphate. The GPD1 may be classified as EC 1.1.1.8.

The external mitochondria NADH dehydrogenase may be an enzyme classified as EC. 1.6.5.9 or EC. 1.6.5.3. The NADH dehydrogenase may be a type II NADH:ubiquinone oxidoreductase. The NADH dehydrogenase may be located on an external surface of an internal mitochondrial membrane, which is disposed towards the cytoplasm. The NADH dehydrogenase may be an enzyme that catalyzes an oxidation of a cytosolic NADH into NAD+. The NADH dehydrogenase may re-oxidize the cytosolic NADH formed according to the process described above. The NADH dehydrogenase may provide the cytosolic NADH to a mitochondrial respiratory chain. The NADH dehydrogenase may be Nde1, Nde2, or a combination thereof. The NADH dehydrogenase may be distinguished from an internal mitochondrial NADH dehydrogenase (NDI1), which acts inside the mitochondria.

The polypeptide that converts acetyl-CoA into ethanol may be Adh. The Adh may be an enzyme that reversibly converts acetyl CoA into ethanol along with an oxidation of NADH into NAD+. The Adh may be an enzyme classified as EC.1.1.1.1. The gene encoding for the polypeptide that converts acetyl CoA into ethanol may have a gene ID of 12753141. The gene may be adhE, which is found in E. coli and codes for an NADH-linked Adh.

The polypeptide that converts oxaloacetate into malate may be an enzyme that catalyzes the conversion of oxaloacetate into malate by using the reduction of NAD+ into NADH. The enzyme may be a malate dehydrogenase. The malate dehydrogenase may be an enzyme classified as EC 1.1.1.37.

A polypeptide of the factor that controls aerobic respiration may be ArcA. The ArcA may be a DNA-binding response regulator. The ArcA may be a DNA-binding response regulator of a two component system. The ArcA belongs to a two component (ArcB-ArcA) signal transmission system group and may cooperate with a sensory kinase ArcB, which belongs to the same group, to form a global regulation system that negatively or positively regulates the expression of various operons. The ArcA operates under a microaerobic condition to induce the expression of gene products that permit activities of central metabolic enzymes that are sensitive to low oxygen levels. The deletion of arcA/arcB under the microaerobic condition may increase specific activities of genes such as ldh, icd, gltA, mdh, and gdh gene.

Inactivation or reduced activity of an additional gene from the yeast cell deleted of the ku80 gene may be performed by a homologous recombination method known in the art.

According to another aspect of the present disclosure, provided is a method of producing a biochemical, the method including: providing a yeast cell having inactivated or reduced activity of the Ku80 polypeptide; inserting a gene that participates in a biochemical biosynthesis pathway in the yeast cell; culturing the yeast cell; and retrieving the biochemical from cultured products obtained therefrom.

The biochemical and the gene that participates in the biochemical biosynthesis pathway are as described above.

The culturing may be performed in a medium containing a carbonaceous source, for example, glucose. The medium used for culturing the yeast cell may be any general medium suitable for the growth of host cells, such as a minimum or a complex medium including suitable supplements.

The medium used for the culturing may be a medium that satisfies requirements of a specific yeast cell. The medium may be selected from the group consisting of a carbonaceous source, a nitrogen source, a salt, a trace element, and a combination thereof.

To obtain a biochemical from the genetically manipulated (i.e. recombinant) yeast cell, the culture condition may be suitably adjusted. The cell is cultured under an aerobic condition for proliferation thereof. Thereafter, the cell is cultured under an aerobic condition or a microaerobic condition to produce lactate.

The expression “culture condition” refers to a condition for culturing a yeast cell. The culture condition may be, for example, a carbon source and a nitrogen source or an oxygen condition used by the yeast cell. The carbon source usable by the yeast cell includes a monosaccharide, a disaccharide, or a polysaccharide. For example, glucose, fructose, mannose, or galactose may be used. The nitrogen source usable by the yeast cell may be an organic nitrogen compound or an inorganic nitrogen compound. For example, the nitrogen source may be an amino acid, an amide, an amine, a nitrogen salt, or an ammonium salt. The oxygen condition for culturing the yeast cell includes an aerobic condition at a normal oxygen partial pressure, a hypoxic condition including about 0.1% to about 10% oxygen in the atmosphere, or an anaerobic condition free of oxygen. A metabolic pathway may be modified according to a carbonaceous source and a nitrogen source that are actually usable by the yeast cell.

The biochemical may be separated from cultured products by using a method known in the art. Such separation method includes centrifugation, filtration, ion exchange chromatography, or crystallization. For example, the cultured products may be centrifuged at a low speed to remove a biomass, and a supernatant obtained therefrom may be separated through ion exchange chromatography.

### EXAMPLE 1

### Preparation of a Strain having Inactivated or Reduced Activities of Ku80

1.1 Preparation of a ku80 Gene Deletion Cassette

To delete a ku80 gene by using a homologous recombination method, a gene deletion vector was prepared as follows:

In a genomic DNA of kluyveromyces marxianus (KCTC17555, also known as ‘Km05’), primers of SEQ ID NO. 9 and 10 were used to amplify a 5′-UTR region to obtain an amplification product (hereinafter, ‘PCR1 amplification product’) of SEQ ID NO. 11. Also, primers of SEQ ID NO. 12 and 13 were used to amplify a 3′-UTR region to obtain an amplification product of SEQ ID NO. 14 (hereinafter, ‘PCR2 amplification product’).

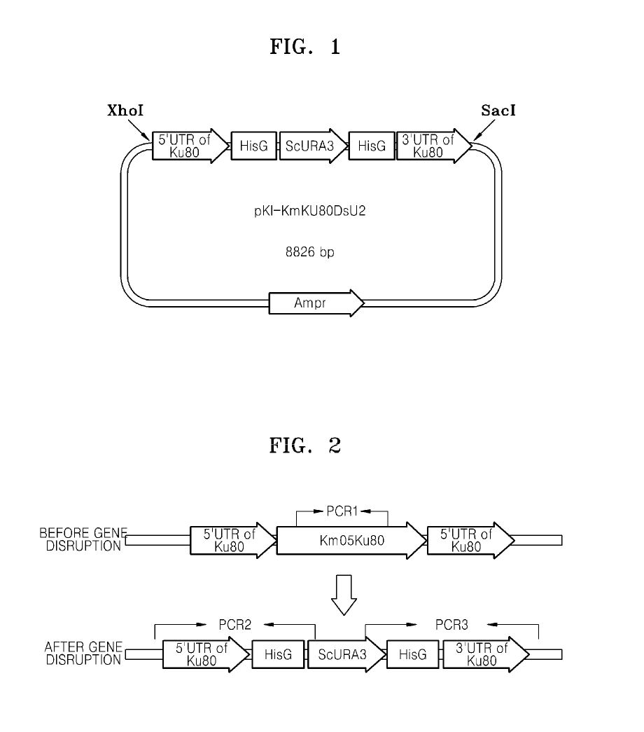

Thereafter, a pKI vector (Samsung Electronics) of SEQ ID NO. 61, including the PCR1 amplification product, an ampicillin resistance gene, a multiple cloning site, and an ScURA3 gene, was excised with an Xhol/EcoRI restriction enzyme, and a product obtained therefrom was ligated and cloned in the pKI vector to prepare a pKI-KmKU80DsU1 vector. Thereafter, the pKI-KmKU80DsU1 vector and the PCR2 amplification products were excised by using a BamHI/Sacl restriction enzyme, and a product obtained therefrom was ligated and cloned in the pKI-KmKU80DsU1 vector to prepare a pKI-KmKU80DsU2 vector. FIG. 1 is a schematic view of a pKI-KmKU80DsU2 vector including a ku80 gene deletion cassette. The pKI-KmKU80DsU2 vector was treated with the Xhol/Sacl restriction enzyme to use a DNA fragment of SEQ ID NO. 15 (size of 5,956 bp) as a ku80 gene deletion cassette.

1.2 Preparation of a ku80 Gene Deleted K. marxianus Strain

A mutant strain, in which a ku80 gene was deleted from K. marxianus, (KCTC17555) was prepared by using a transformation method described below.

K. marxianus (KCTC17555) was inoculated in a YPD (1% yeast extract, 2% bacto peptone, and 2% glucose) liquid medium, cultured for 14 hours at a temperature of 37° C. in a shaking incubator, cultured in 50 ml of the YPD liquid medium for 3 hours at a temperature of 37° C. in a shaking incubator until optical density at 600 nanometers (OD₆₀₀) reached about 0.3, and when OD₆₀₀ reached about 1.0, cells were centrifuged at 4,000 rpm for 5 minutes, which were suspended and washed in a TE solution (0.01 M Tris-HCl pH 7.5 and 1 mM EDTA pH8.0). Thereafter, the cells were centrifuged at 4,000 rpm for 5 minutes to resuspend the cells in a lithium acetate/TE solution (100 mM lithium acetate pH 7.5, 0.01M Tris-HCl pH 7.5, and 1 mM EDTA pH8.0), which were then divided in an amount of 100 ul.

To delete a ku80 gene, the ku80 deletion cassette prepared in Example 1.1, 40% polyethylene glycol, and the lithium acetate/TE solution mixture solution at the above-mentioned concentration were mixed with a single-stranded carrier DNA, and then cultured in a shaker incubator at a temperature of 30° C. for 30 minutes. Thereafter, 70 ul of DMSO (Sigma) was added thereto to react in a bath at a temperature of 42° C. for 25 minutes and a culture medium was spread onto a plate selecting a synthetic complete without uracil (SC-URA: 0.67% yeast nitrogen base without amino acid, 2% glucose, amino acid mixture without uracil). A colony formed on the plate was inoculated in a YPD medium, and genomic DNA was extracted by using an STES buffer solution (0.5M NaCI, 0.2M Tris-HCl (pH 7.6), 0.01M EDTA, 1% SDS), beads, and phenol/chloroform/isoamyl alcohol.

The genomic DNA of the separated mutant strain was used as a template for PCR1, PCR2, and PCR3 to confirm the deletion of the ku80 gene, and electrophoresis was performed on each of the DNA amplification products obtained therefrom to confirm the deletion of the ku80 gene. FIG. 2 is a schematic view of PCR1, PCR2, and PCR3 for deletion of a ku80 gene. PCR1 included the use of primers of SEQ ID NOs. 16 and 17, PCR2 included the use of primers of SEQ ID NO. 18 and 19, and PCR3 included the use of primers of SEQ ID NO. 20 and 21. As a result, K. marxianus (KCTC17555)Δku80+URA3 strain was obtained.

Also, for an additional gene deletion by using the ku80 gene deletion vector, URA3 gene, which was a selection marker inserted in the ku80 deletion cassette to prepare the K. marxianus (KCTC17555)Δku80 strain, was deleted as follows:

K. marxianus (KCTC17555) Δku80+URA3 was inoculated in 2 ml of a YPD liquid medium to culture the same for 14 hours at a temperature of 37° C., the cultured strain was diluted such that OD₆₀₀ was 1, and 50 ul (5 x 10⁵ cells) thereof was smeared on a synthetic complete (SC) medium in which 0.5 g/L of FOA (5-FluoroOrotic Acid) and uracil were added in a concentration of 90 mg/l. At this point, the grown colony was respread onto a YPD complete medium and an SC-URA minimal medium to obtain a strain in which the URA3 gene was deleted again.

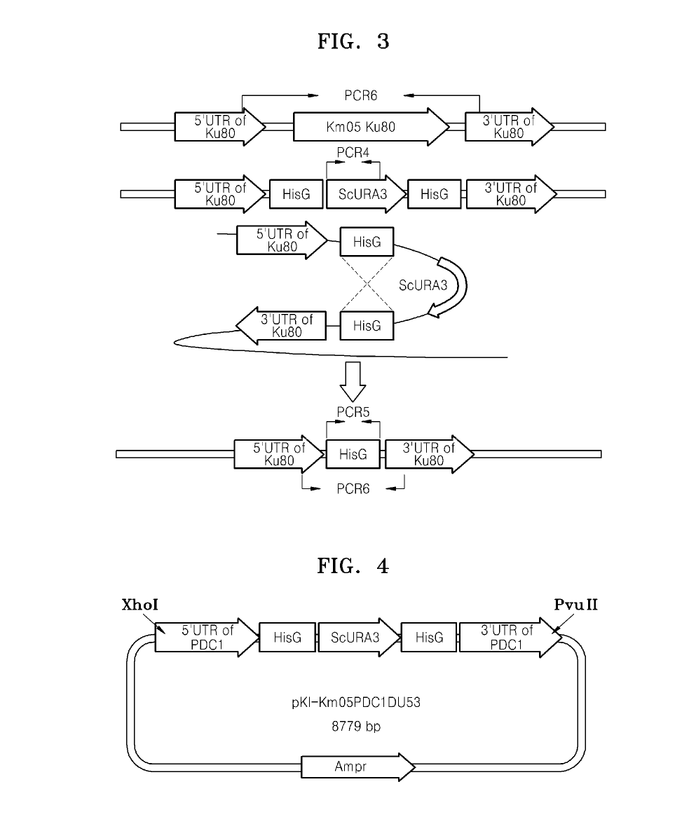

Ten colonies (URA3 pop-out strain) grown on the plate were selected, patched onto a 5-FOA plate, and, at the same time, inoculated into a YPD liquid medium to isolate the genomic DNA from the strain by using the method described above. The genomic DNA of the URA3 pop-out strain was used as a template for PCR4, PCR5, and PCR6 to confirm the deletion of URA3, and PCR products obtained therefrom were electrophoresed to confirm the deletion of the URA3. PCR4 included the use of primers of SEQ ID NO. 22 and 23, PCR5 included the use of primers of SEQ ID NO. 24 and 25, and PCR6 included the use of primers of SEQ ID NO. 26 and 27. FIG. 3 is a PCR amplification region for confirmation of deletion of URA3. As a result, K. marxianus (KCTC17555)Δku80 was obtained.

### EXAMPLE 2

### Deletion of pdcl Gene by using a Strain in which Ku80 was Inactivated

2.1 Preparation of a pdcl Gene Deletion Cassette

In a strain deleted of Ku80, a gene deletion vector was prepared to delete a pdcl gene by using a homologous recombination method, as follows:

In a genomic DNA of K. marxianus (KCTC17555), primers of SEQ ID NO. 28 and 29 were used to amplify a 5′-UTR region to obtain an amplification product of SEQ ID NO. 30 (hereinafter, ‘PCR3 amplification product’) and primers of SEQ ID NO. 31 and 32 were used to amplify a 3′-UTR region to obtain an amplification product of SEQ ID NO. 33 (hereinafter ‘PCR4 amplification product’).

Thereafter, a pKI vector including the PCR3 amplification product, an ampicillin resistance gene, a multiple cloning site, and an ScURA3 gene was excised by using an Xhol/BglII restriction enzyme, and a product obtained therefrom was ligated and cloned in the pKI vector prepare a pKI-Km05PDC1 DU5 vector. Thereafter, the pKI-Km05PDC1 DU5 vector was excised by using an Xbal/EcolCRI restriction enzyme, the PCR4 amplification product was excised by using an Xbal restriction enzyme, and a product obtained therefrom was ligated and cloned in the pKI-Km05PDC1 DU5 vector to prepare a pKI-Km05PDC1DU53 vector. FIG. 4 is a schematic view of a pKI-Km05PDC1DU53 vector including the pdcl gene deletion cassette. The pKI-Km05PDC1 DU53 vector was treated with an Xhol/Pvull restriction enzyme and a DNA fragment of SEQ ID NO. 34 (size of 5,895 bp) was used as a pdcl gene deletion cassette.

2.2 Deletion of a pdcl Gene by using a Strain in which Ku80 was Inactivated

To confirm genetic manipulation efficiency, K. marxianus (KCTC17555)Δku80 prepared in Example 1.2 was used to delete a pdcl gene, and as a control group, wild-type K. marxianus (KCTC17555) was deleted of the pdcl gene in the same manner. The K. marxianus (KCTC17555)Δku80 prepared in Example 1.2 and the wild-type K. /marxianus (KCTC17555) strain were used for transformation of about 10 ug of linear DNA by using the method described in Example 1.2.

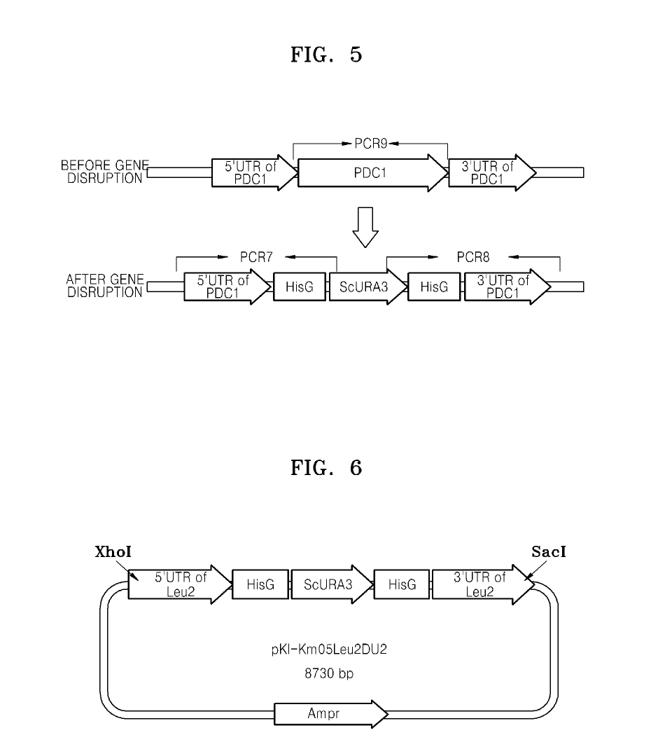

Genomic DNA was extracted from the colonies formed on the plate by using the method described in Example 1.2, PCR7, PCR8, and PCR9 were performed to confirm the deletion of the pdcl gene, and DNA amplification products obtained therefrom were electrophoresed to select mutant strains deleted of the pdcl gene. FIG. 5 is a schematic view of PCR for confirmation of deletion of a pdcl gene. PCR7 included the use of primers of SEQ ID NO. 35 and 36, PCR8 included the use of primers of SEQ ID NO. 37 and 38, and PCR9 included the use of primers of SEQ ID NO. 39 and 40. As a result, K. marxianus (KCTC17555)Δpdc1 and K. marxianus (KCTC17555)Δku80Δpdc1 were obtained.

Table 1 shows deletion efficiencies of the pdcl gene in K. marxianus deleted of the ku80 gene and the wild-type K. marxianus. As shown in Table 1, the pdcl gene deletion efficiency of the K. marxianus deleted of the ku80 gene was 38.4%, which was higher than the pdcl gene deletion efficiency of the wild-type K. marxianus (KCTC17555), which was 16.7%.

TABLE 1

| | Strain | |
|---|---|---|
| | Wild-type | K. marxianus |
| | K. marxianus | (KCTC17555) |
| | (KCTC17555) | (Δ ku80) |
| Colony transformed with a pdc1 | 12 colonies | 13 colonies |
| gene deletion cassette | | |
| Colony in which pdc1 gene was | 2 colonies | 5 colonies |
| confirmed | | |
| pdc1 gene deletion efficiency | 16.7% | 38.4% |

### EXAMPLE 3

### Deletion of a leu2 Gene by using a Strain in which ku80 was Inactivated

3.1 Preparation of a lue2 Gene Deletion Cassette

To delete a lue2 gene through a homologous recombination method, a lue2 gene deletion vector was prepared as follows: In a genomic DNA of K. marxianus (KCTC17555) strain, primers of SEQ ID NO. 41 and 42 were used to amplify a 5′-UTR region to obtain an amplification product of SEQ ID NO. 43 (hereinafter ‘PCR5 amplification product’) and primers of SEQ ID NO. 44 and 45 were used to amplify a 3′-UTR region to obtain an amplification product of SEQ ID NO. 46 (hereinafter, ‘PCR6 amplification product’).

Thereafter, a pKI vector including the PCR5 amplification product, an ampicillin resistance gene, a multiple cloning site, and ScURA3gene was excised by using an Xhol/BglIl restriction enzyme, and a product obtained therefrom was ligated and cloned in the pKI vector to prepare a pKI-Km05Leu2DU1 vector. Thereafter, the pKI-Km05Leu2DU1 vector and the PCR6 amplification product were excised by using a Spel/Sacl restriction enzyme, and a product obtained therefrom was ligated and cloned in the pKI-Km05Leu2DU1 vector to prepare a pKI-Km05Leu2DU2 vector. FIG. 6 is a schematic view of a pKI-Km05LEU2DU2 vector including a leu2 gene deletion cassette. The pKI-Km05Leu2DU2 vector was treated with Xhol/Sacl to use a DNA fragment having a SEQ ID NO. 47 (size of 5,860 bp) as a leu2 gene deletion cassette.

3.2 Deletion of a leu2 Gene by using a Strain in which Ku80 was Inactivated

To confirm genetic manipulation efficiency, K. marxianus (KCTC17555)Δku80 prepared in Example 1.2 was used to delete a leu2 gene and as a control group, a wild-type K. marxianus (KCTC17555) was deleted of the leu2 gene in the same manner.

K. marxianus (KCTC17555)Δku80 prepared in Example 1.2 and a wild-type K. marxianus (KCTC17555) strain were used for transformation of the deletion cassette prepared in Example 1.2 and about 10 ug of linear DNA by using the method described in Example 3.1.

The colonies formed on the plate were primarily selected in an SC-URA medium and then sequentially inoculated in SC and SC(-Leu) minimal media to analyze growth. In greater detail, an auxotroph plate free of leucine, SC(-Leu), which was used to confirm the deletion of the leu2 gene, was used to culture the mutant strain. As a result, K. marxianus (KCTC17555)Δleu2 and K. marxianus (KCTC17555)Δku80Δleu2, which cannot be grown in a medium lacking leucine, were obtained.

Table 2 shows growth analysis results in the medium as well as the leu2 gene deletion efficiency of K. marxianus deleted of a ku80 gene and wild-type K. marxianus. As shown in Table 2, the leu2 gene deletion efficiency of K. marxianus deleted of the ku80 gene was 76.2%, which was higher than the leu2 gene deletion efficiency of the wild-type K. marxianus (KCTC17555), which was 4.8%.

TABLE 2

| | Strain | |
|---|---|---|
| | Wild-type | K. marxianus |
| | K. marxianus | (KCTC17555) |
| | (KCTC17555) | (Δ ku80) |
| Colonies transformed with a lue2 | 21 colonies | 21 colonies |
| gene deletion cassette | | |
| Colonies in which lue2 gene | 1 colony | 16 colonies |
| deletion was confirmed | | |
| lue2 gene deletion efficiency | 4.8% | 76.2% |

### EXAMPLE 4

### Deletion of a pdc5 Gene by using a Strain in which Ku80 was Inactivated

4.1 Preparation of a pdc5 Gene Deletion Cassette

To delete the pdc5 gene by using a homologous recombination method, a pdc5 gene deletion vector was prepared as follows:

In a genomic DNA of a K. marxianus (KCTC17555) strain, primers of SEQ ID NO. 48 and 49 were used to amplify a 5′-UTR region to obtain an amplification product having SEQ ID NO. 50 (hereinafter ‘PCR7 amplification product’) and primers of SEQ ID NO. 51 and 52 were used to amplify a 3′-UTR region to obtain an amplification product having SEQ ID NO. 53 (hereinafter, ‘PCR8 amplification product’).

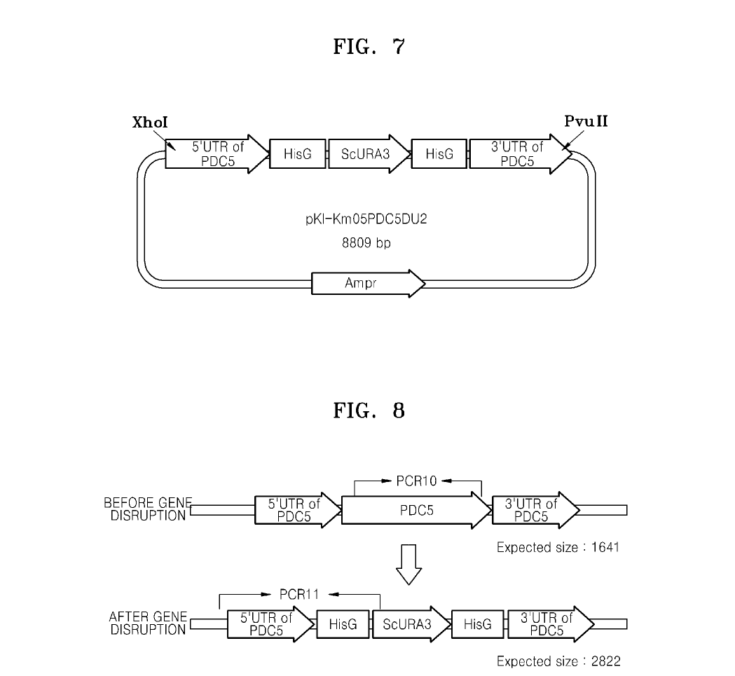

Thereafter, a pKI vector including the PCR7 amplification product, an ampicillin resistance gene, a multiple cloning site, and ScURA3gene was excised by using an Xhol/EcoRl restriction enzyme, and a product obtained therefrom was ligated and cloned in the pKI vector to prepare a pKI-Km05PDC6DU1 vector. Thereafter, the pKI-Km05PDC6DU1 vector and the PCR8 amplification product were excised by using a Spel/Sacl restriction enzyme, and a product obtained therefrom was ligated and cloned in the pKI-Km05PDC6DU1 vector to prepare a pKI-Km05PDC6DU2 vector. FIG. 7 is a schematic view of a pKI-Km05PDC5DU2 vector including a pdc5 gene deletion cassette. The pKI-Km05PDC6DU2 vector was treated with Xhol/Pvull to use a DNA fragment of SEQ ID NO. 54 (size of 5,939 bp) as a pdc5 gene deletion cassette.

4.2 Deletion of a PDC5 Gene by using a Strain in which Ku80 was Inactivated

To confirm genetic manipulation efficiency, K. marxianus (KCTC17555) Δku80 prepared in Example 1.2 was used to delete a pdc5 gene and as a control group, a wild-type K. marxianus (KCTC17555) was deleted of the pdc5 gene in the same manner.

The K. marxianus (KCTC17555)Δku80 prepared in Example 1.2 and the wild-type K. marxianus (KCTC17555) strain were used for transformation of the deletion cassette prepared in Example 4.1 and 10 ug of linear DNA in the same manner as described in Example 1.2.

The colonies formed on the plate were primarily selected for candidate strains that did not yield an amplification product, by using primers of SEQ ID NO. 55 and 56 that bind to a portion of a C-terminal of pdc5 during PCR.

Thereafter, to reconfirm mutant strains deleted of a pdc5 gene, genomic DNA was extracted from mutant strains lacking the amplification of the about 150 by DNA fragment in the same manner as in Example 1.2 and primers that bind to a portion of the C-terminal described above were reused to perform PCR and then electrophoresis.

Thereafter, the genomic DNA of candidate strains without the amplification of the C-terminal were used to perform PCR10 and PCR11, and DNA amplification products obtained therefrom were identified by using electrophoresis. FIG. 8 is a schematic view of a PCR amplification region for confirmation of deletion of a pdc5 gene. PCR10 included the use of primers of SEQ ID NO. 57 and 58 and PCR11 included the use of primers of SEQ ID NO. 59 and 60. As a result, K. marxianus (KCTC17555)Δpdc5 was not obtained and only K. marxianus (KCTC17555) Δku80Δpdc5 was obtained.

Table 3 shows the pdc5 gene deletion efficiency of K. marxianus deleted of a ku80 gene and wild-type K. marxianus. As shown in Table 3, the pdc5 gene deletion efficiency of the ku80 gene was 13%, which was higher than the pdc5 gene removal efficiency of wild-type K. marxianus (KCTC17555), which was 0%.

TABLE 3

| | Strain | |
|---|---|---|
| | Wild-type | K. marxianus |
| | K. marxianus | (KCTC17555) |
| | (KCTC17555) | (Δku80) |
| Colonies transformed with PDC5 | 71 colonies | 23 colonies |
| gene deletion cassette | | |
| Colonies confirmed to have PDC5 | 0 colonies | 3 colonies |
| gene deletion | | |
| PDC5 gene deletion efficiency | 0% | 13% |

As described above, provided is a recombinant yeast cell having enhanced genetic manipulation efficiency. According to a method of preparing a recombinant yeast cell deleted of a ku80 gene of the present disclosure, provided is a recombinant yeast cell having enhanced genetic manipulation efficiency. According to a yeast cell having enhanced genetic manipulation efficiency of the present disclosure, a desired gene may be efficiently manipulated. According to a method of producing a biochemical of the present disclosure, the biochemical may be efficiently produced.

It should be understood that the exemplary embodiments described therein should be considered in a descriptive sense only and not for purposes of limitation. Descriptions of features or aspects within each embodiment should typically be considered as available for other similar features or aspects in other embodiments.

While one or more embodiments of the present disclosure have been described with reference to the figures, it will be understood by those of ordinary skill in the art that various changes in form and details may be made therein without departing from the spirit and scope of the present disclosure as defined by the following claims.

It should be understood that the exemplary embodiments described herein should be considered in a descriptive sense only and not for purposes of limitation. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A recombinant yeast cell, wherein the recombinant yeast cell has a Crabtree-negative phenotype and a deletion or disruption mutation of a gene encoding Ku80 polypeptide.

2. The recombinant yeast cell of claim 1, wherein the recombinant yeast cell has reduced or eliminated Ku80 polypeptide activity.

3. The recombinant yeast cell of claim 1, wherein the recombinant yeast cell is Kluyveromyces marxianus, Kluyveromyces lactis, or Kluyveromyces waltii.

4. The recombinant yeast cell of claim 1, wherein the recombinant yeast cell is Kluyveromyces marxianus.

5. The recombinant yeast cell of claim 1, wherein the recombinant yeast cell have improved efficiency in genetic manipulation.

6. The recombinant yeast cell of claim 1, wherein the recombinant yeast cell has reduced non-homologous end-joining activity.

7. The recombinant yeast cell of claim 1, wherein the yeast cell comprises a deletion or disruption mutation of a gene encoding the polypeptide converting pyruvate into acetaldehyde, a gene encoding the 3-isopropylmalate dehydrogenase polypeptide, or a combination thereof.

8. The recombinant yeast cell of claim 1, further comprising an exogenous gene that participates in a biochemical biosynthesis pathway, wherein the biochemical is 1,4-butanediol, 4-hydroxy-butyraldehyde, lactate, 1,2-propanediol, 1,3-propanediol, or ethylene glycol.

9. A method of providing a recombinant yeast cell with improved efficiency in genetic manipulation, the method comprising introducing a mutation in a Ku80 gene of the Crabtree-negative yeast cell, wherein the mutation reduces or eliminates Ku80 polypeptide activity and non-homologous end-joining activity in the yeast cell.

10. The method of claim 9, wherein the mutation is a deletion or disruption mutation in the Ku80 gene.

11. The method of claim 9, wherein introducing a mutation in a Ku80 gene of the Crabtree-negative yeast cell comprises:
providing a Ku80 gene deletion cassette, wherein the cassette comprises a gene-specific homologous region that has a sequence identity with a portion of the Ku80 gene sufficient to facilitate homologous recombination of the cassette with the portion of the Ku80 gene; and
inserting the cassette into the Crabtree-negative yeast cell, whereby at least a portion of the Ku80 gene is deleted.

12. The method of claim 11, the method further comprising culturing the yeast cell, and selecting a transformed yeast cell from the cultures.

13. The method of claim 9, wherein the recombinant yeast cell has a Crabtree-negative phenotype.

14. The method of claim 9, wherein the recombinant yeast cell is Kluyveromyces marxianus, Kluyveromyces lactis, or Kluyveromyces waltii.

15. The method of claim 9, wherein the recombinant yeast cell is Kluyveromyces marxianus.

16. The method of claim 9, further comprising inserting a gene that participates in a biochemical biosynthesis pathway into the recombinant yeast cell.

17. A method of producing a biochemical, the method comprising:
providing the recombinant yeast cell of claim 1;
inserting a gene that participates in a biochemical biosynthesis pathway in the yeast cell;
culturing the resulting recombinant yeast cell; and
retrieving a biochemical from cultured products obtained therefrom.

18. The method of claim 17, wherein the biochemical is 1,4-butanediol, 4-hydroxy-butyraldehyde, lactate, 1,2-propanediol, 1,3-propanediol, or ethylene glycol.
